(19) United States
(12) Patent Application Publication
(10) Pub. No.: US 2020/0045974 A1
(43) Pub. Date: Feb. 13, 2020

(54) Substituted Oxadiazoles for Combating Phytopathogenic Fungi

(71) Applicant: BASF SE, Ludwigshafen am Rhein (DE)

(72) Inventors: Violeta TERTERYAN-SEISER, Ludwigshafen (DE); Wassilios GRAMMENOS, Ludwigshafen (DE); Maria Angelica QUINTERO PALOMAR, Limburgerhof (DE); Ian Robert CRAIG, Ludwigshafen (DE); Christine WIEBE, Ludwigshafen (DE); Tobias MENTZEL, Limburgerhof (DE); Marcus FEHR, Limburgerhof (DE); Ana ESCRIBANO CUESTA, Ludwigshafen (DE); Christian Harald WINTER, Navi Mumbai (IN); Jan Klaas LOHMANN, Ludwigshafen (DE); Bernd MUELLER, Ludwigshafen (DE); Erica CAMBEIS, Ludwigshafen (DE); Thomas GROTE, Ludwigshafen (DE); Michael SEET, Ludwigshafen (DE)

(21) Appl. No.: 16/500,536

(22) PCT Filed: Mar. 29, 2018
(86) PCT No.: PCT/EP2018/058060

§ 371 Date: Oct. 3, 2019

(30) Foreign Application Priority Data
Apr. 7, 2017 (EP) 17165495.7

(51) Int. Cl. A01N 43/82; C07D 271/06

(52) U.S. Cl. CPC A01N 43/82; C07D 271/06

(57) ABSTRACT

The present invention relates to novel trifluoromethyloxadiazoles of the formula I, or the N-oxides, or the agriculturally useful salts thereof; and to their use for controlling phytopathogenic fungi; and to a method for combating phytopathogenic harmful fungi, which process comprises treating the fungi, the plants, the soil or seeds to be protected against fungal attack, with an effective amount of at least one compound of the formula I, or an N-oxide, or an agriculturally acceptable salt thereof; and to agrochemical compositions comprising at least one compound of the formula I; and to agrochemical compositions further comprising seeds.

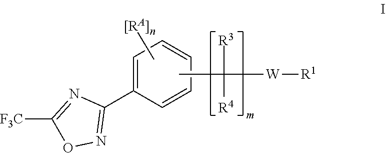

The present invention relates to novel trifluoromethyloxadiazoles of the formula I, or the N-oxides, or the agriculturally useful salts thereof; and to their use for controlling phytopathogenic fungi; and to a method for combating phytopathogenic harmful fungi, which process comprises treating the fungi, the plants, the soil or seeds to be protected against fungal attack, with an effective amount of at least one compound of the formula I, or an N-oxide, or an agriculturally acceptable salt thereof; and to agrochemical compositions comprising at least one compound of the formula I; and to agrochemical compositions further comprising seeds.

EP 276432 A2 relates to 3-phenyl-5-trifluoromethyloxadiazole derivatives and to their use to combat phytopathogenic microorganisms. WO 2015/185485 A1, WO 2017/055469 A1 and WO 2017/055473 A1 describe other derivatives of trifluoromethyloxadiazoles and their use to combat phytopathogenic microorganisms. WO 97/30047 A1 describes certain trifluoromethyloxadiazole analogues with fungicidal activity.

In many cases, in particular at low application rates, the fungicidal activity of known fungicidal compounds is unsatisfactory. Based on this, it was an objective of the present invention to provide compounds having improved activity and/or a broader activity spectrum against phytopathogenic fungi. This objective is achieved by the oxadiazoles of the formula I and/or their agriculturally useful salts for controlling phytopathogenic fungi.

The compounds described herein differ from compounds known in the prior art in the constitution of the group —(CR³R⁴)ₘ—W—R¹, in particular with regard to the nature of R¹.

Accordingly, the present invention relates to compounds of the formula I, or the N-oxides, or the agriculturally acceptable salts thereof

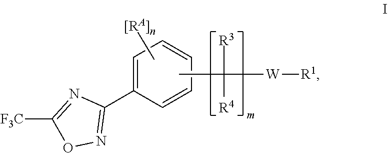

wherein:
R^{A} is independently selected from the group consisting of halogen, cyano, C₁-C₆-alkyl, C₁-C₆-haloalkyl, C₁-C₆-alkoxy and C₁-C₆-haloalkoxy;
n is 0, 1 or 2;
R¹ is a bicyclic carbocycle of the formula R^{a}

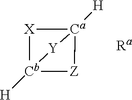

wherein
C^{a} and C^{b} are bridgehead carbon atoms;
X is a direct single bond or a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—;
Y and Z independently of each other are a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—;
   or R¹ is a tricyclic carbocycle of the formula R^{b}

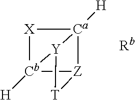

wherein
C^{a} and C^{b} are bridgehead carbon atoms;
X is a direct single bond or a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—;
Y and Z independently of each other are a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—; and wherein groups Y and Z are attached to the bridgehead carbon atoms C^{a} and C^{b};
T is a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—; and wherein the group T is attached to one carbon atom in each of the groups Y and Z;
and with the proviso that, if R¹ is a tricyclic carbocycle of the formula R^{b}, wherein X is a direct single bond or a divalent group —CH₂—, the groups T and Z independently of each other are a divalent group selected from the group consisting of —CH₂—CH₂—, —(CH₂)₃—, —(CH₂)₄—, —CH═CH—, —CH₂—CH═CH—, —CH═CH—CH₂— and —CH═CH—CH═CH—;
and wherein the groups R^{a} or R^{b} are connected to the group W through one of the ring carbon atoms;
and wherein R¹ is unsubstituted or substituted with 1, 2, 3, 4 or up to the maximum possible number of radicals selected from the group consisting of oxo, hydroxy, halogen, C₁-C₃-alkyl, C₁-C₃-haloalkyl, C₃-C₆-cycloalkyl, vinylidene and dichlorovinylidene;
W is #1-(C═O)—NR²-#2, #1-NR²—(C═O)-#2, #1-(C═S)—NR²-#2, #1-NR²—(C═S)-#2, #1-S(═O)ₚ—NR²-#2 or #1-NR²—S(═O)ₚ-#2; wherein #1 denotes the position, which is attached to the group —CR³R⁴— or, if m is 0, to the phenyl group, and #2 denotes the position, which is attached to R¹;
p is 0, 1 or 2;
R² is hydrogen, C₁-C₆-alkyl, C₂-C₆-alkenyl, C₂-C₆-alkynyl, C₁-C₆-alkoxy, C₃-C₈-cycloalkyl, C₃-C₈-cycloalkenyl, C₃-C₈-cycloalkyl-C₁-C₄-alkyl, phenyl-C₁-C₄-alkyl, phenyl, C(═O)—(C₁-C₆-alkyl) or C(═O)—(C₁-C₆-alkoxy); and wherein any of the aliphatic or cyclic groups are unsubstituted or substituted with 1, 2, 3 or up to the maximum possible number of identical or different radicals selected from the group consisting of halogen, hydroxy, cyano, C₁-C₆-alkyl, C₁-C₆-alkoxy and C₃-C₈-cycloalkyl;
m is 0 or 1;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy;
or R³ and R⁴ together with the carbon atom to which they are bound form a monocyclic 3- to 5-membered saturated heterocycle or saturated carbocycle; and wherein the saturated heterocycle includes beside one or more carbon atoms no heteroatoms or 1 or 2 heteroatoms independently selected from N, O and S as ring member atoms; and wherein the heterocycle or the carbocycle is unsubstituted or substituted 1, 2, 3, 4 or up to the maximum possible number of identical or different radicals selected from the group consisting of halogen, cyano and C₁-C₂-alkyl;
with the exception of compounds of the formula I, wherein
m is 0;
W is #1-NR²—(C═O)-#2, #1-NR²—(C═S)-#2 or #1-NR²—S(═O)ₚ-#2, wherein the variables p and R² are defined as herein above for compounds of the formula I; and
R¹ is a bicyclic carbocycle of the formula R^{a}.

Agriculturally acceptable salts of the compounds of the formula I encompass especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may be substituted with one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of acceptable acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound I with an acid of the corresponding anion, preferably of hydrochloric acid, hydrobromic acid, sulfuric acid, phosphoric acid or nitric acid.

Compounds of the formula I can exist as one or more stereoisomers. The various stereoisomers include enantiomers, diastereomers, atropisomers arising from restricted rotation about a single bond of asymmetric groups and geometric isomers. They also form part of the subject matter of the present invention. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, e.g. a racemate, individual stereoisomers, or as an optically active form.

Compounds of the formula I can be present in different crystal modifications whose biological activity may differ. They also form part of the subject matter of the present invention.

In respect of the variables, the embodiments of the intermediates obtained during preparation of compounds I correspond to the embodiments of the compounds of formula I. The term “compounds I” refers to compounds of formula I.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₆-alkyl” refers to a straight-chained or branched saturated hydrocarbon group having 1 to 6 carbon atoms, for example methyl, ethyl, propyl, 1-methylethyl, butyl, 1-methylpropyl, 2-methylpropyl, and 1,1-dimethylethyl.

The term “C₂-C₆-alkenyl” refers to a straight-chain or branched unsaturated hydrocarbon radical having 2 to 6 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond, such as ethynyl, 1-propynyl, 2-propynyl (propargyl), 1-butynyl, 2-butynyl, 3-butynyl, 1-methyl-2-propynyl.

The term “C₁-C₆-haloalkyl” refers to a straight-chained or branched alkyl group having 1 to 6 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl.

The term “C₁-C₆-alkoxy” refers to a straight-chain or branched alkyl group having 1 to 6 carbon atoms (as defined above) which is bonded via an oxygen, at any position in the alkyl group, for example methoxy, ethoxy, n-propoxy, 1-methylethoxy, butoxy, 1-methylpropoxy, 2-methylpropoxy or 1,1-dimethylethoxy.

The term “vinylidene” refers to a group ═CH₂, the term “dichlorovinylidene” refers to a group ═CCl₂.

The term “phenyl-C₁-C₄-alkyl” refers to alkyl having 1 to 4 carbon atoms (as defined above), wherein one hydrogen atom of the alkyl radical is replaced by a phenyl radical.

The term “C₃-C₈-cycloalkyl” refers to monocyclic saturated hydrocarbon radicals having 3 to 8 carbon ring members such as cyclopropyl (C₃H₅), cyclobutyl, cyclopentyl, cyclohexyl, cycloheptyl or cyclooctyl.

The terms “C(═O)—(C₁-C₄-alkyl) or C(═O)—(C₁-C₄-alkoxy)” refer to a radical which is attached through the carbon atom of the —C(═O)— group as indicated by the number valence of the carbon atom.

The term “aliphatic” refers to compounds or radicals composed of carbon and hydrogen and which are non-aromatic compounds. An “alicyclic” compound or radical is an organic compound that is both aliphatic and cyclic. They contain one or more all-carbon rings which may be either saturated or unsaturated, but do not have aromatic character.

The terms “cyclic moiety” or “cyclic group” refer to a radical which is an alicyclic ring or an aromatic ring, such as, for example, phenyl or heteroaryl.

The term “and wherein any of the aliphatic or cyclic groups are unsubstituted or substituted with . . . ” refers to aliphatic groups, cyclic groups and groups, which contain an aliphatic and a cyclic moiety in one group, such as in, for example, C₃-C₈-cycloalkyl-C₁-C₄-alkyl; therefore a group which contains an aliphatic and a cyclic moiety both of these moieties may be substituted or unsubstituted independently of each other.

The term “phenyl” refers to an aromatic ring systems including six carbon atoms (commonly referred to as benzene ring.

The term “wherein R¹ is connected to the group W through one of the ring carbon atoms of the groups R^{a} or R^{b}” in the context of this invention means that R¹ is attached to the group W through one carbon atom of the groups R^{a} or R^{b}, which includes any carbon atom of X, Y, Z, T and the bridgehead carbon atoms C^{a} and C^{b}, thereby substituting a hydrogen atom on said carbon atom.

In respect of the variables, the embodiments of the intermediates correspond to the embodiments of the compounds I. Preference is given to those compounds I and, where applicable, also to compounds of all subformulae provided herein, e. g. formulae I.1, I.2, I.3, I.4, or the generic groups of compounds I.1a and I.3a, wherein the variables W, p, R¹, R², R³, R⁴, R^{A}, m and n have independently of each other or more preferably in combination (any possible combination of 2 or more substituents as defined herein) the following meanings:

In a preferred embodiment R^{A} is independently selected from the group consisting of halogen, C₁-C₆-alkyl or C₃-C₈-cycloalkyl. In another preferred embodiment R^{A} is independently selected from the group consisting of halogen, methyl or ethyl. More preferably R^{A} is independently selected from the group consisting of halogen, in particular R^{A} is fluorine.

In one aspect of the invention n is 0, 1 or 2, preferably n is 0 or 1. In a particularly preferred aspect n is 0.

In one aspect of the invention R¹ is a bicyclic carbocycle of the formula R^{a}

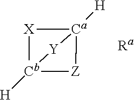

wherein
C^{a} and C^{b} are bridgehead carbon atoms;
X is a direct single bond or a divalent group selected from the group consisting of —CH₂— or —CH₂—CH₂—;
Y and Z independently of each other are a divalent group selected from the group consisting of —CH₂— or —CH₂—CH₂—;
   and wherein the group R^{a} is connected to the group W through one of the ring carbon atoms; and wherein R^{a} is unsubstituted or substituted with 1, 2, 3, 4 or up to the maximum possible number of radicals selected from the group consisting of oxo, hydroxy, halogen, C₁-C₃-alkyl.

In another aspect of the invention R¹ is a tricyclic carbocycle of the formula R^{b}

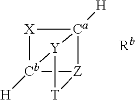

wherein
C^{a} and C^{b} are bridgehead carbon atoms;
X is a direct single bond or a divalent group selected from the group consisting of —CH₂— or —CH₂—CH₂—;
Y and Z independently of each other are a divalent group selected from the group consisting of —CH₂— or —CH₂—CH₂—; and wherein groups Y and Z are attached to the bridgehead carbon atoms C^{a} and C^{b};
T is a divalent group selected from the group consisting of —CH₂— or —CH₂—CH₂—; and wherein the group T is attached to one carbon atom in each of the groups Y and Z;
   and with the proviso that, if X is a direct single bond or a divalent group —CH₂—, the groups T and Z independently of each other are a divalent group —CH₂—CH₂—;
   and wherein the group R^{b} is connected to the group W through one of the ring carbon atoms; and wherein R^{b} is unsubstituted or substituted with 1, 2, 3, 4 or up to the maximum possible number of radicals selected from the group consisting of oxo, hydroxy, halogen, C₁-C₃-alkyl.

In a further aspect R¹ is a bicyclic or tricyclic carbocycle selected from the group consisting of radicals R¹.1 to R¹.31 below; wherein each radical may be connected to the group W through one of the ring carbon atoms by substitution of one hydrogen atom; and wherein R¹ is unsubstituted or substituted with 1, 2, 3, 4 or up to the maximum possible number of radicals selected from the group consisting of oxo, hydroxy, halogen and C₁-C₃-alkyl.

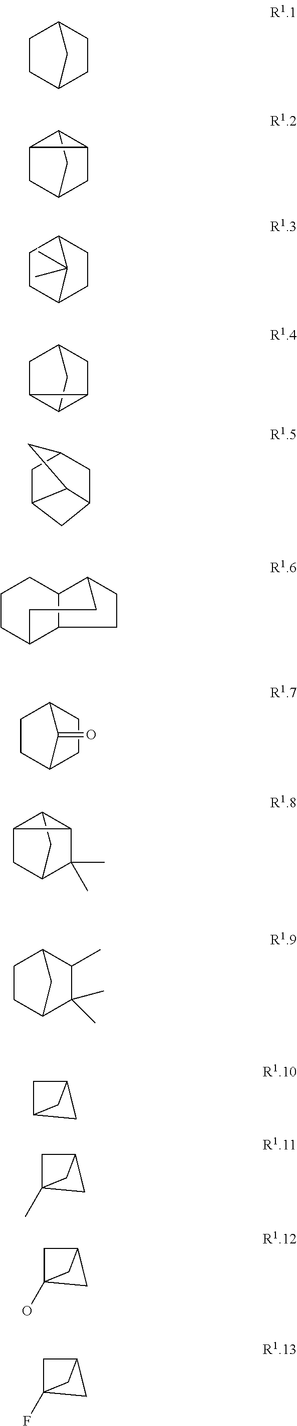

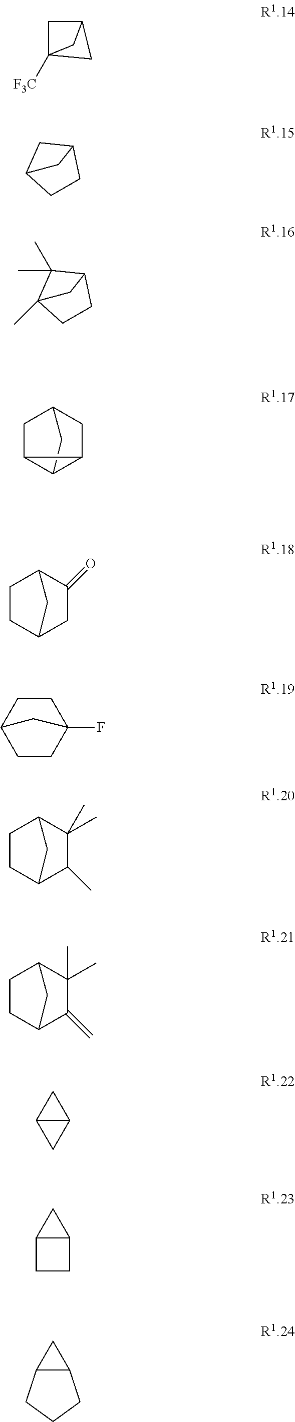

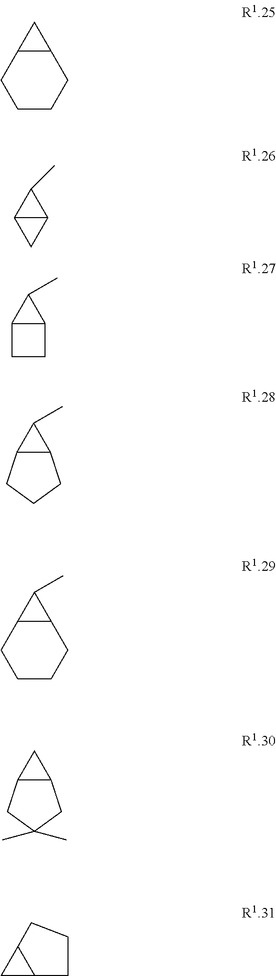

In another aspect R¹ is a bicyclic carbocycle selected from the group consisting of radicals R¹.10, R¹.15, R¹.22, R¹.23, R¹.24, R¹.25, R¹.26, R¹.27 and R¹.31; preferably R¹.10, R¹.15, R¹.22 and R¹.23; and wherein each radical may be connected to the group W through one of the ring carbon atoms by substitution of one hydrogen atom; and wherein R¹ is unsubstituted or substituted with 1, 2, 3, 4 or up to the maximum possible number of radicals selected from the group consisting of oxo, hydroxy, halogen and C₁-C₃-alkyl.

Particularly preferred radicals R¹ are selected from the group consisting of R¹.32 to R¹.57 below, which are further unsubstituted, and wherein “# C” indicates the carbon atom, which is attached to the group W.

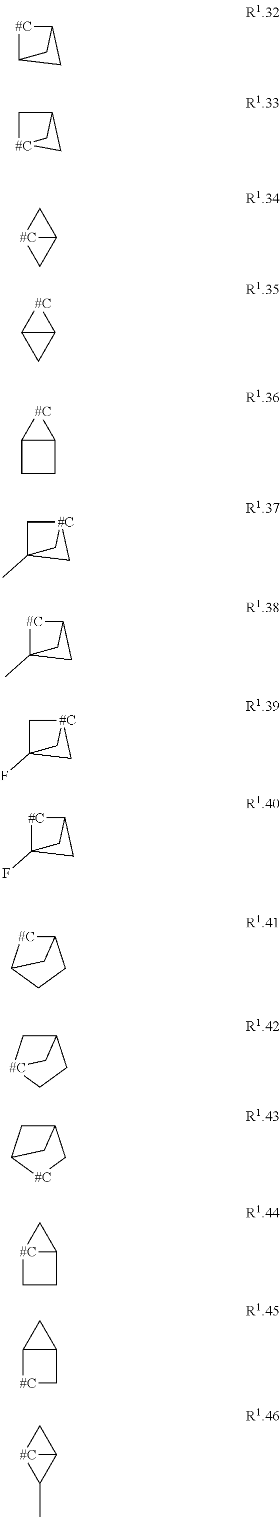

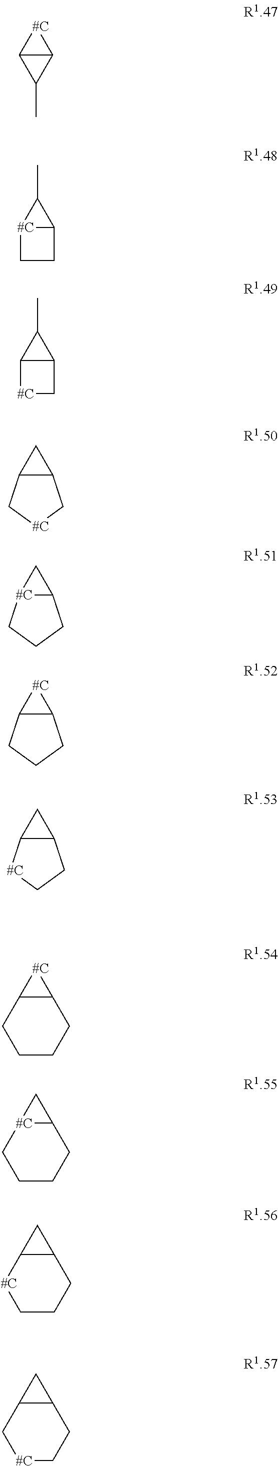

In one aspect of the invention R² is hydrogen, C₁-C₆-alkyl, C₁-C₆-alkoxy, C₂-C₆-alkenyl, ethynyl, propargyl, C₃-C₈-cycloalkyl, C₃-C₈-cycloalkenyl, C₃-C₈-cycloalkyl-C₁-C₄-alkyl or phenyl; and wherein any of the aliphatic or cyclic groups are unsubstituted or substituted with 1, 2, 3, 4 or up to the maximum possible number of identical or different radicals selected from the group consisting of halogen, cyano, C₁-C₆-alkyl and C₁-C₆-alkoxy; more preferably from halogen, in particular the radical is fluorine.

In another aspect R² is hydrogen, C₁-C₆-alkyl, C₂-C₆-alkenyl, propargyl, C₃-C₈-cycloalkyl, C₃-C₈-cycloalkenyl, C₃-C₈-cycloalkyl-C₁-C₄-alkyl or phenyl; and wherein any of the aliphatic or cyclic groups are unsubstituted or substituted with 1, 2, 3, 4 or up to the maximum possible number of identical or different radicals selected from the group consisting of halogen, cyano, C₁-C₆-alkyl and C₁-C₆-alkoxy; more preferably from halogen, in particular the radical is fluorine.

In a preferred aspect of the invention R² is hydrogen, C₁-C₆-alkyl, C₁-C₆-alkenyl, propargyl, C₃-C₈-cycloalkyl, C₃-C₈-cycloalkyl-C₁-C₄-alkyl or phenyl; and wherein any of the aliphatic or cyclic groups are unsubstituted or substituted with 1, 2, 3, 4 or up to the maximum possible number of identical or different radicals selected from the group consisting of halogen or C₁-C₆-alkyl, in particular fluorine.

In another aspect R² is hydrogen, C₁-C₆-alkyl, C₁-C₆-alkoxy, C₂-C₆-alkenyl, propargyl, C₃-C₈-cycloalkyl or C₃-C₈-cycloalkyl-C₁-C₄-alkyl.

In a further aspect R² is hydrogen, C₁-C₆-alkyl, C₂-C₆-alkenyl or propargyl.

In yet another aspect R² is hydrogen, methyl, ethyl, iso-propyl, cyclopropyl, cyclopropyl-CH₂—, vinyl, allyl, phenyl, 4-F-phenyl or 2-F-phenyl.

In a more preferred aspect of the invention R² is hydrogen, methyl, ethyl, n-propyl, iso-propyl, cyclopropyl, cyclopropyl-CH₂—, vinyl or allyl.

In a further preferred aspect of the invention R² is hydrogen, methyl, ethyl, n-propyl, iso-propyl, cyclopropyl or vinyl.

In a particularly preferred aspect R² is hydrogen, methy or ethyl.

In a further preferred aspect R² is C₁-C₆-alkoxy.

In a further aspect R² is hydrogen, C₁-C₆-alkyl, C₂-C₆-alkenyl, C₃-C₈-cycloalkyl or C₃-C₈-cycloalkyl-C₁-C₄-alkyl; in particular hydrogen, methyl, ethyl, iso-propyl, cyclopropyl, cyclopropyl-CH₂—, vinyl or allyl; more particularly hydrogen, methyl or ethyl; and R¹ is a bicyclic carbocycle of the formula R^{a}

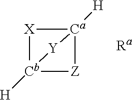

wherein
C^{a} and C^{b} are bridgehead carbon atoms;
X is a direct single bond or a divalent group selected from the group consisting of —CH₂— or —CH₂—CH₂—;
Y and Z independently of each other are a divalent group selected from the group consisting of —CH₂— or —CH₂—CH₂—;
   and wherein R¹ is connected to the group W through one of the ring carbon atoms of the groups R^{a}; and wherein R¹ is unsubstituted or substituted with 1, 2, 3, 4 or up to the maximum possible number of radicals selected from the group consisting of oxo, hydroxy, halogen, C₁-C₃-alkyl.

In yet another embodiment R² is hydrogen, C₁-C₆-alkyl, C₂-C₆-alkenyl or C₃-C₈-cycloalkyl; in particular hydrogen, methyl, ethyl, iso-propyl, cyclopropyl, cyclopropyl-CH₂—, vinyl or allyl; more particularly hydrogen, methyl or ethyl; and R¹ is a bicyclic carbocycle selected from the group consisting of radicals R¹.10, R¹.15, R¹.22 and R¹.23; and wherein each radical R¹ may be connected to the group W through one of the ring carbon atoms by substitution of one hydrogen atom; and wherein R¹ is unsubstituted or substituted with 1, 2, 3, 4 or up to the maximum possible number of radicals selected from the group consisting of oxo, hydroxy, halogen and C₁-C₃-alkyl.

In a preferred aspect of the invention R² is hydrogen, C₁-C₆-alkyl, C₂-C₆-alkenyl, C₃-C₈-cycloalkyl or C₃-C₈-cycloalkyl-C₁-C₄-alkyl; in particular hydrogen, methyl, ethyl, iso-propyl, cyclopropyl, cyclopropyl-CH₂—, vinyl or allyl; more particularly hydrogen, methyl or ethyl; and R¹ is a bicyclic carbocycle selected from the group consisting of radicals R¹.10, R¹.15, R¹.22 and R¹.23; and wherein each radical R¹ may be connected to the group W through one of the ring carbon atoms by substitution of one hydrogen atom; and wherein R¹ is unsubstituted or substituted with 1, 2, 3, 4 or up to the maximum possible number of radicals selected from the group consisting of oxo, hydroxy, halogen and C₁-C₃-alkyl.

In one embodiment the invention relates to compounds of the formula I, wherein R³ and R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy.

In one embodiment the invention relates to compounds of the formula I, wherein R³ and R⁴ independently of each other are selected from the group consisting of hydrogen, halogen, C₁-C₆-alkyl or C₁-C₆-haloalkyl.

In another embodiment the invention relates to compounds of the formula I, wherein R³ and R⁴ independently of each other are selected from the group consisting of hydrogen or C₁-C₄-alkyl; preferably hydrogen, methyl or ethyl.

In a further embodiment R³ and R⁴ are independently of each other hydrogen, fluorine, methyl or trifluoromethyl. In another aspect R³ and R⁴ are both hydrogen. In a further aspect R³ is hydrogen and R⁴ is methyl. In yet another aspect R³ and R⁴ are both methyl. In a further aspect R³ and R⁴ are both fluorine. In one aspect R³ is hydrogen and R⁴ is trifluoromethyl. In one embodiment R³ and R⁴ are both trifluoromethyl.

In one embodiment R³ and R⁴ together with the carbon atom to which they are bound form a monocyclic 3- to 5-membered saturated heterocycle or saturated carbocycle; and wherein the saturated heterocycle includes beside one or more carbon atoms no heteroatoms or 1 or 2 heteroatoms independently selected from N, O and S as ring member atoms; and wherein the heterocycle or the carbocycle is unsubstituted or substituted 1, 2, 3, 4 or up to the maximum possible number of identical or different radicals selected from the group consisting of halogen, cyano and C₁-C₂-alkyl.

In another embodiment R³ and R⁴ together with the carbon atom to which they are bound form a 3- or 4-membered carbocylic ring; and wherein the carbocylic ring is unsubstituted.

In another aspect R³ and R⁴ together with the carbon atom to which they are bound form a cyclopropyl ring, wherein the cyclopropyl ring is unsubstituted.

In still another embodiment R³ and R⁴ together with the carbon atom to which they are bound form a saturated 3-membered heterocycle; wherein the heterocycle includes beside two carbon atoms one heteroatom selected from N, O and S as ring member atoms; and wherein the heterocycle is unsubstituted.

In one embodiment W is #1-(C═O)—NR²-#2, #1-(C═S)—NR²-#2 or #1-S(═O)ₚ—NR²-#2; wherein #1 denotes the position, which is attached to the phenyl group, and #2 denotes the position, which is attached to R¹.

In one embodiment of the invention m is 1. In another embodiment m is 0.

In a preferred embodiment m is 1 and W is #1-NR²—(C═O)-#2, #1-NR²—(C═S)-#2 or #1-NR²—S(═O)ₚ-#2, wherein p is 0, 1 or 2; preferably p is 2; wherein #1 denotes the position, which is attached to the group —CR³R⁴— and #2 denotes the position, which is attached to R¹.

In another preferred embodiment m is 0 and W is #1-(C═O)—NR²-#2, #1-NR²—(C═O)-#2, #1-(C═S)—NR²-#2, #1-NR²—(C═S)-#2, #1-S(═O)ₚ—NR²-#2 or #1-NR²—S(═O)ₚ-#2; more preferably #1-(C═O)—NR²-#2 or #1-(C═S)—NR²-#2; in particular #1-(C═O)—NR²-#2; wherein #1 denotes the position, which is attached to the phenyl group and #2 denotes the position, which is attached to R¹.

In one embodiment the invention relates to compounds of the formulae I.1, I.2, I.3 or I.4, or the N-oxides, or the agriculturally acceptable salts thereof,

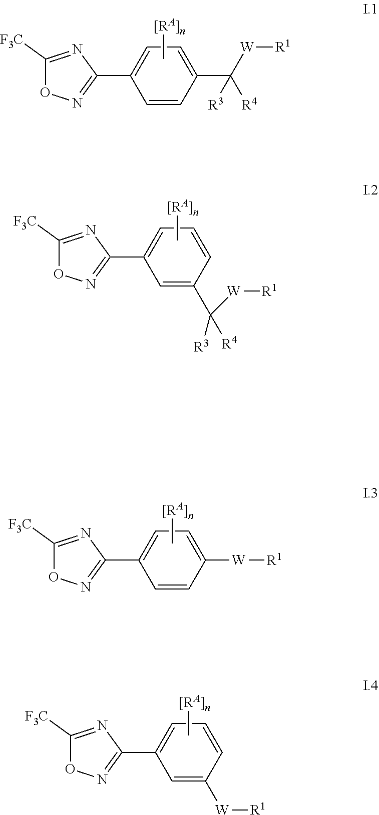

wherein n is 0 or 1, and wherein the meaning of the variables W, R^{A}, R¹, R³, R⁴ are as defined or preferably defined herein for compounds of the formula I.

In a further embodiment the invention relates to the group of compounds I.1a of formula I.1, or the N-oxides, or the agriculturally acceptable salts thereof, wherein:
R^{A} is halogen, cyano, C₁-C₆-alkyl, C₁-C₆-haloalkyl, C₁-C₆-alkoxy or C₁-C₆-haloalkoxy;
n is 0 or 1;
R¹ is a bicyclic carbocycle of the formula R^{a}

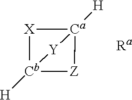

wherein
C^{a} and C^{b} are bridgehead carbon atoms;
X is a direct single bond or a divalent group selected from the group consisting of —CH₂— or —CH₂—CH₂—;
Y and Z independently of each other are a divalent group selected from the group consisting of —CH₂— or —CH₂—CH₂—;
and wherein R^{a} is connected to the group W through one of the ring carbon atoms; and
wherein R^{a} is unsubstituted or substituted with 1, 2, 3, 4 or up to the maximum possible number of radicals selected from the group consisting of oxo, hydroxy, halogen and C₁-C₃-alkyl;
W is #1-(C═O)—NR²-#2, #1-NR²—(C═O)-#2, #1-(C═S)—NR²-#2, #1-NR²—(C═S)-#2, #1-S(═O)ₚ—NR²-#2 or #1-NR²—S(═O)ₚ-#2; wherein #1 denotes the position, which is attached to the group —CR³R⁴— and #2 denotes the position, which is attached to R¹;
R² is hydrogen, C₁-C₆-alkyl, C₂-C₆-alkenyl, C₂-C₆-alkynyl, C₁-C₆-alkoxy, C₃-C₈-cycloalkyl, C₃-C₈-cycloalkenyl, C₃-C₈-cycloalkyl-C₁-C₄-alkyl, phenyl-C₁-C₄-alkyl, phenyl, C(═O)—(C₁-C₆-alkyl) or C(═O)—(C₁-C₆-alkoxy); and wherein any of the aliphatic or cyclic groups are unsubstituted or substituted with 1, 2, 3 or up to the maximum possible number of identical or different radicals selected from the group consisting of halogen, hydroxy, cyano, C₁-C₆-alkyl, C₁-C₆-alkoxy and C₃-C₈-cycloalkyl;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy;
or R³ and R⁴ together with the carbon atom to which they are bound form a cyclopropyl ring.

In a further embodiment the invention relates to the group of compounds I.1a, wherein n is 0, and wherein R³ and R⁴ are independently selected from the group consisting of hydrogen, halogen, C₁-C₆-alkyl and C₁-C₆-haloalkyl; or R³ and R⁴ together with the carbon atom to which they are bound form a cyclopropyl ring. In yet another embodiment the invention relates to the group of compounds 1.1a, wherein R³ and R⁴ are hydrogen.

In a further embodiment the invention relates to the group of compounds I.3a of formula 1.3, or the N-oxides, or the agriculturally acceptable salts thereof, wherein:
R^{A} is halogen, cyano, C₁-C₆-alkyl, C₁-C₆-haloalkyl, C₁-C₆-alkoxy or C₁-C₆-haloalkoxy;
n is 0 or 1;
R¹ is a bicyclic carbocycle of the formula R^{a}

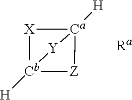

wherein
C^{a} and C^{b} are bridgehead carbon atoms;
X is a direct single bond or a divalent group selected from the group consisting of —CH₂— or —CH₂—CH₂—;
Y and Z independently of each other are a divalent group selected from the group consisting of —CH₂— or —CH₂—CH₂—;
and wherein R^{a} is connected to the group W through one of the ring carbon atoms; and
wherein R^{a} is unsubstituted or substituted with 1, 2, 3, 4 or up to the maximum possible number of radicals selected from the group consisting of oxo, hydroxy, halogen and C₁-C₃-alkyl;
W is #1-(C═O)—NR²-#2, #1-(C═S)—NR²-#2 or #1-S(═O)ₚ—NR²-#2; wherein #1 denotes the position, which is attached to the phenyl group, and #2 denotes the position, which is attached to R¹;
R² is hydrogen, C₁-C₆-alkyl, C₂-C₆-alkenyl, C₂-C₆-alkynyl, C₁-C₆-alkoxy, C₃-C₈-cycloalkyl, C₃-C₈-cycloalkenyl, C₃-C₈-cycloalkyl-C₁-C₄-alkyl, phenyl-C₁-C₄-alkyl, phenyl, C(═O)—(C₁-C₆-alkyl) or C(═O)—(C₁-C₆-alkoxy); and wherein any of the aliphatic or cyclic groups are unsubstituted or substituted with 1, 2, 3 or up to the maximum possible number of identical or different radicals selected from the group consisting of halogen, hydroxy, cyano, C₁-C₆-alkyl, C₁-C₆-alkoxy and C₃-C₈-cycloalkyl.

In a further embodiment the invention relates to the group of compounds I.3a, wherein n is 0.

Another embodiment of the invention relates to compounds of the formulae 1.1 or 1.2, wherein n is 0; W is #1-(C═O)—NR²-#2, #1-NR²—(C═O)-#2, #1-(C═S)—NR²-#2 or #1-NR²—(C═S)-#2; wherein #1 denotes the position, which is attached to the group —CR³R⁴— and #2 denotes the position, which is attached to R¹; R³ and R⁴ are independently selected from the group consisting of hydrogen, fluorine and methyl; or R³ and R⁴ together with the carbon atom to which they are bound form a cyclopropyl ring; and wherein R¹ and R² are as defined or preferably defined herein.

Another embodiment of the invention relates to compounds of the formulae 1.1 or 1.2, wherein n is 0; W is #1-(C═O)—NR²-#2, #1-NR²—(C═O)-#2, #1-(C═S)—NR²-#2 or #1-NR²—(C═S)-#2; wherein #1 denotes the position, which is attached to the group —CR³R⁴— and #2 denotes the position, which is attached to R¹; R³ and R⁴ are independently selected from the group consisting of hydrogen, fluorine and methyl; or R³ and R⁴ together with the carbon atom to which they are bound form a cyclopropyl ring; and wherein R¹ and R² are as defined or preferably defined herein.

Another embodiment of the invention relates to compounds of the formulae 1.3 or 1.4, wherein n is 0; W is #1-(C═O)—NR²-#2 or #1-(C═S)—NR²-#2; wherein #1 denotes the position, which is attached to the phenyl ring and #2 denotes the position, which is attached to R¹; and wherein R¹ and R² are as defined or preferably defined herein.

According to one embodiment, the present invention relates to compounds of the formulae I.A, I.B, I.C, I.D. I.E, I.F, I.G, I.H, I.J, I.K, I.L, I.M, I.N, I.O, I.P, I.Q, I.R, I.S, I.T, I.O and I.V and to their use for controlling phytopathogenic fungi, wherein the variables R¹ and R² in compounds of the formulae I.A, I.B, I.C, I.D. I.E, I.F, I.G, I.H, I.J, I.K, I.L, I.M, I.N, I.O, I.P, I.Q, I.R, I.S, I.T, I.O and I.V are as defined or preferably defined herein.

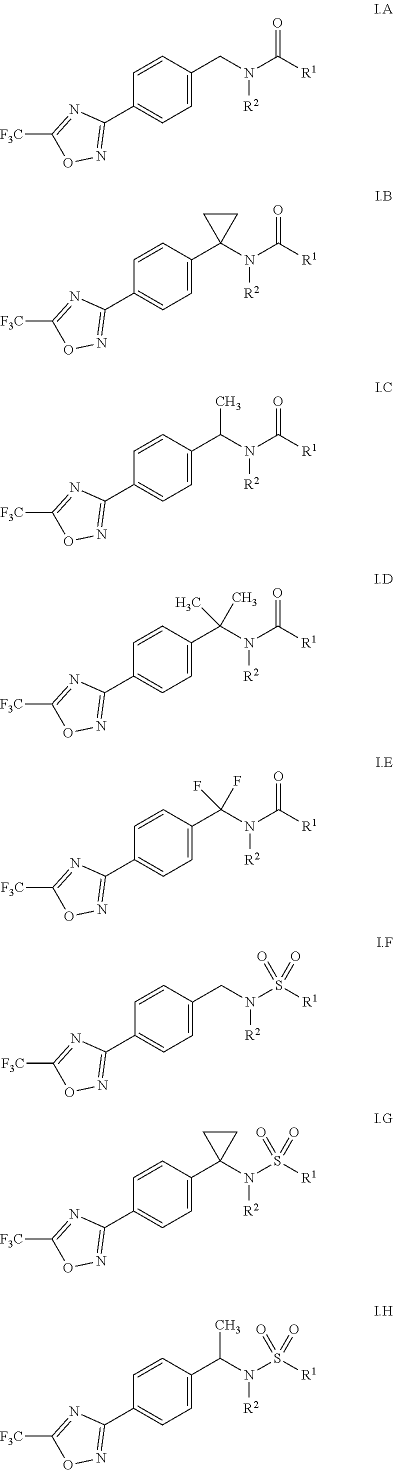

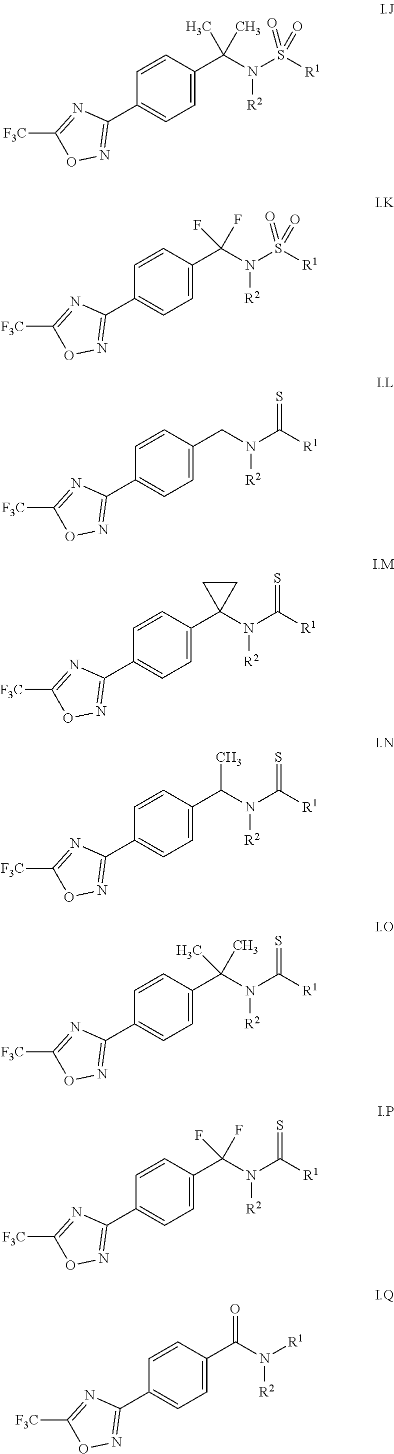

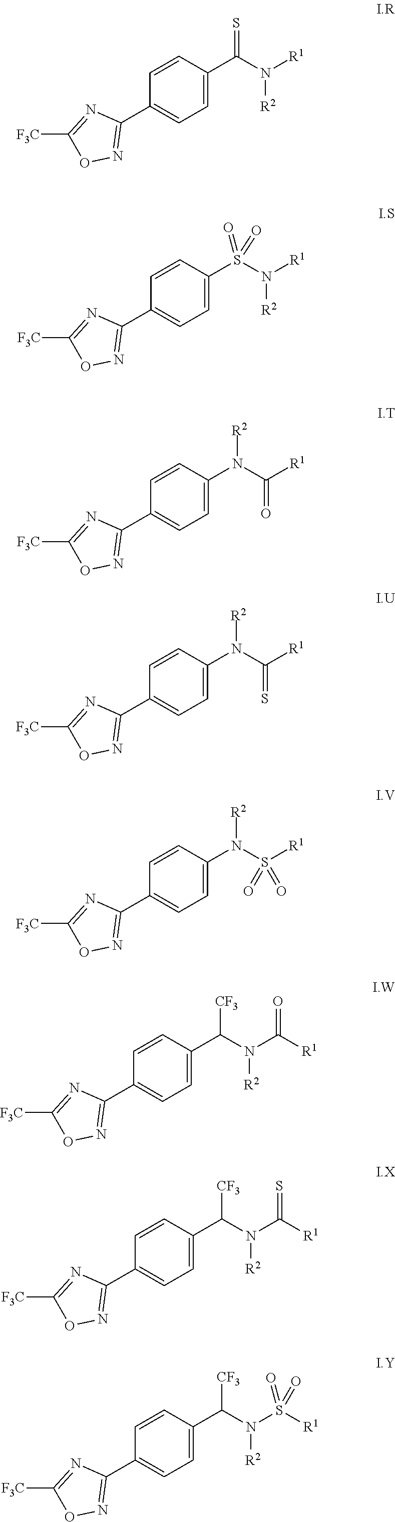

Preference is given to compounds of the formula I, which are compiled in Tables 1 to 24 below, and which may be used according to the invention.

Table 1: Compounds of the formula I.A, in which R¹ and R² for each individual compound corresponds in each case to one line A-1 to A-208 of Table A (compounds I.A.A-1 to I.A.A-208). This means, for example, that a compound of formula I.A, wherein R¹ is radical R¹.35 as defined herein, and wherein R² is hydrogen (corresponding to the definition A-4 in Table A) is named I.A.A-4

Table 2: Compounds of the formula I.B, in which R¹ and R² for each individual compound corresponds in each case to one line A-1 to A-208 of Table A (compounds I.B.A-1 to I.B.A-208). This means, for example, that a compound of formula I.B, wherein R¹ is radical R¹.35 as defined herein, and wherein R² is allyl (corresponding to the definition A-114 in Table A) is named I.B.A-114

Table 3: Compounds of the formula I.C, in which R¹ and R² for each individual compound corresponds in each case to one line A-1 to A-208 of Table A (compounds I.C.A-1 to I.C.A-208)

Table 4: Compounds of the formula I.D, in which R¹ and R² for each individual compound corresponds in each case to one line A-1 to A-208 of Table A (compounds I.D.A-1 to I.D.A-208).

Table 5: Compounds of the formula I.E, in which R¹ and R² for each individual compound corresponds in each case to one line A-1 to A-208 of Table A (compounds I.E.A-1 to I.E.A-208).

Table 6: Compounds of the formula I.F, in which R¹ and R² for each individual compound corresponds in each case to one line A-1 to A-208 of Table A (compounds I.F.A-1 to I.F.A-208).

Table 7: Compounds of the formula I.G, in which R¹ and R² for each individual compound corresponds in each case to one line A-1 to A-208 of Table A (compounds I.G.A-1 to I.G.A-208).

Table 8: Compounds of the formula I.H, in which R¹ and R² for each individual compound corresponds in each case to one line A-1 to A-208 of Table A (compounds I.H.A-1 to I.H.A-208)

Table 9: Compounds of the formula I.J, in which R¹ and R² for each individual compound corresponds in each case to one line A-1 to A-208 of Table A (compounds I.J.A-1 to I.J.A-208).

Table 10: Compounds of the formula I.K, in which R¹ and R² for each individual compound corresponds in each case to one line A-1 to A-208 of Table A (compounds I.K.A-1 to I.K.A-208).

Table 11: Compounds of the formula I.L, in which R¹ and R² for each individual compound corresponds in each case to one line A-1 to A-208 of Table A (compounds I.L.A-1 to I.L.A-208).

Table 12: Compounds of the formula I.M, in which R¹ and R² for each individual compound corresponds in each case to one line A-1 to A-208 of Table A (compounds I.M.A-1 to I.M.A-208).

Table 13: Compounds of the formula I.N, in which R¹ and R² for each individual compound corresponds in each case to one line A-1 to A-208 of Table A (compounds I.N.A-1 to I.N.A-208)

Table 14: Compounds of the formula 1.0, in which R¹ and R² for each individual compound corresponds in each case to one line A-1 to A-208 of Table A (compounds I.O.A-1 to I.O.A-208).

Table 15: Compounds of the formula I.P, in which R¹ and R² for each individual compound corresponds in each case to one line A-1 to A-208 of Table A (compounds I.P.A-1 to I.P.A-208).

Table 16: Compounds of the formula I.Q, in which R¹ and R² for each individual compound corresponds in each case to one line A-1 to A-208 of Table A (compounds I.Q.A-1 to I.Q.A-208).

Table 17: Compounds of the formula I.R, in which R¹ and R² for each individual compound corresponds in each case to one line A-1 to A-208 of Table A (compounds I.R.A-1 to I.R.A-208).

Table 18: Compounds of the formula I.S, in which R¹ and R² for each individual compound corresponds in each case to one line A-1 to A-208 of Table A (compounds I.S.A-1 to I.S.A-208).

Table 19: Compounds of the formula I.T, in which R¹ and R² for each individual compound corresponds in each case to one line A-1 to A-208 of Table A (compounds I.T.A-1 to I.T.A-208)

Table 20: Compounds of the formula I.U, in which R¹ and R² for each individual compound corresponds in each case to one line A-1 to A-208 of Table A (compounds I.U.A-1 to I.U.A-208).

Table 21: Compounds of the formula I.V, in which R¹ and R² for each individual compound corresponds in each case to one line A-1 to A-208 of Table A (compounds I.V.A-1 to I.V.A-208).

Table 22: Compounds of the formula I.W, in which R¹ and R² for each individual compound corresponds in each case to one line A-1 to A-208 of Table A (compounds I.W.A-1 to I.W.A-208).

Table 23: Compounds of the formula I.X, in which R¹ and R² for each individual compound corresponds in each case to one line A-1 to A-208 of Table A (compounds I.X.A-1 to I.X.A-208).

Table 24: Compounds of the formula I.Y, in which R¹ and R² for each individual compound corresponds in each case to one line A-1 to A-208 of Table A (compounds I.Y.A-1 to I.Y.A-208).

TABLE A

| No. | R¹ | R² |
|---|---|---|
| A-1 | R¹.32 | hydrogen |
| A-2 | R¹.33 | hydrogen |
| A-3 | R¹.34 | hydrogen |
| A-4 | R¹.35 | hydrogen |
| A-5 | R¹.36 | hydrogen |
| A-6 | R¹.37 | hydrogen |
| A-7 | R¹.38 | hydrogen |
| A-8 | R¹.39 | hydrogen |
| A-9 | R¹.40 | hydrogen |
| A-10 | R¹.41 | hydrogen |
| A-11 | R¹.42 | hydrogen |
| A-12 | R¹.43 | hydrogen |
| A-13 | R¹.44 | hydrogen |
| A-14 | R¹.45 | hydrogen |
| A-15 | R¹.46 | hydrogen |
| A-16 | R¹.47 | hydrogen |
| A-17 | R¹.48 | hydrogen |
| A-18 | R¹.49 | hydrogen |
| A-19 | R¹.40 | hydrogen |
| A-20 | R¹.51 | hydrogen |
| A-21 | R¹.52 | hydrogen |
| A-22 | R¹.53 | hydrogen |
| A-23 | R¹.54 | hydrogen |
| A-24 | R¹.55 | hydrogen |
| A-25 | R¹.56 | hydrogen |
| A-26 | R¹.57 | hydrogen |
| A-27 | R¹.32 | methyl |
| A-28 | R¹.33 | methyl |
| A-29 | R¹.34 | methyl |
| A-30 | R¹.35 | methyl |
| A-31 | R¹.36 | methyl |
| A-32 | R¹.37 | methyl |
| A-33 | R¹.38 | methyl |
| A-34 | R¹.39 | methyl |
| A-35 | R¹.40 | methyl |
| A-36 | R¹.41 | methyl |
| A-37 | R¹.42 | methyl |
| A-38 | R¹.43 | methyl |
| A-39 | R¹.44 | methyl |
| A-40 | R¹.45 | methyl |
| A-41 | R¹.46 | methyl |
| A-42 | R¹.47 | methyl |
| A-43 | R¹.48 | methyl |
| A-44 | R¹.49 | methyl |
| A-45 | R¹.40 | methyl |
| A-46 | R¹.51 | methyl |
| A-47 | R¹.52 | methyl |
| A-48 | R¹.53 | methyl |
| A-49 | R¹.54 | methyl |
| A-50 | R¹.55 | methyl |
| A-51 | R¹.56 | methyl |
| A-52 | R¹.57 | methyl |
| A-53 | R¹.32 | ethyl |
| A-54 | R¹.33 | ethyl |
| A-55 | R¹.34 | ethyl |
| A-56 | R¹.35 | ethyl |
| A-57 | R¹.36 | ethyl |
| A-58 | R¹.37 | ethyl |
| A-59 | R¹.38 | ethyl |
| A-60 | R¹.39 | ethyl |
| A-61 | R¹.40 | ethyl |
| A-62 | R¹.41 | ethyl |
| A-63 | R¹.42 | ethyl |
| A-64 | R¹.43 | ethyl |
| A-65 | R¹.44 | ethyl |
| A-66 | R¹.45 | ethyl |
| A-67 | R¹.46 | ethyl |
| A-68 | R¹.47 | ethyl |
| A-69 | R¹.48 | ethyl |
| A-70 | R¹.49 | ethyl |
| A-71 | R¹.40 | ethyl |
| A-72 | R¹.51 | ethyl |
| A-73 | R¹.52 | ethyl |
| A-74 | R¹.53 | ethyl |
| A-75 | R¹.54 | ethyl |
| A-76 | R¹.55 | ethyl |
| A-77 | R¹.56 | ethyl |
| A-78 | R¹.57 | ethyl |
| A-79 | R¹.32 | n-propyl |
| A-80 | R¹.33 | n-propyl |
| A-81 | R¹.34 | n-propyl |
| A-82 | R¹.35 | n-propyl |
| A-83 | R¹.36 | n-propyl |
| A-84 | R¹.37 | n-propyl |
| A-85 | R¹.38 | n-propyl |
| A-86 | R¹.39 | n-propyl |
| A-87 | R¹.40 | n-propyl |
| A-88 | R¹.41 | n-propyl |
| A-89 | R¹.42 | n-propyl |
| A-90 | R¹.43 | n-propyl |
| A-91 | R¹.44 | n-propyl |
| A-92 | R¹.45 | n-propyl |
| A-93 | R¹.46 | n-propyl |
| A-94 | R¹.47 | n-propyl |
| A-95 | R¹.48 | n-propyl |
| A-96 | R¹.49 | n-propyl |
| A-97 | R¹.40 | n-propyl |
| A-98 | R¹.51 | n-propyl |
| A-99 | R¹.52 | n-propyl |
| A-100 | R¹.53 | n-propyl |
| A-101 | R¹.54 | n-propyl |
| A-102 | R¹.55 | n-propyl |
| A-103 | R¹.56 | n-propyl |
| A-104 | R¹.57 | n-propyl |
| A-105 | R¹.32 | iso-propyl |
| A-106 | R¹.33 | iso-propyl |
| A-107 | R¹.34 | iso-propyl |
| A-108 | R¹.35 | iso-propyl |
| A-109 | R¹.36 | iso-propyl |
| A-110 | R¹.37 | iso-propyl |
| A-111 | R¹.38 | iso-propyl |
| A-112 | R¹.39 | iso-propyl |
| A-113 | R¹.40 | iso-propyl |
| A-114 | R¹.41 | iso-propyl |
| A-115 | R¹.42 | iso-propyl |
| A-116 | R¹.43 | iso-propyl |
| A-117 | R¹.44 | iso-propyl |
| A-118 | R¹.45 | iso-propyl |
| A-119 | R¹.46 | iso-propyl |
| A-120 | R¹.47 | iso-propyl |
| A-121 | R¹.48 | iso-propyl |
| A-122 | R¹.49 | iso-propyl |
| A-123 | R¹.40 | iso-propyl |
| A-124 | R¹.51 | iso-propyl |
| A-125 | R¹.52 | iso-propyl |
| A-126 | R¹.53 | iso-propyl |
| A-127 | R¹.54 | iso-propyl |
| A-128 | R¹.55 | iso-propyl |
| A-129 | R¹.56 | iso-propyl |
| A-130 | R¹.57 | iso-propyl |
| A-131 | R¹.32 | vinyl |
| A-132 | R¹.33 | vinyl |
| A-133 | R¹.34 | vinyl |
| A-134 | R¹.35 | vinyl |
| A-135 | R¹.36 | vinyl |
| A-136 | R¹.37 | vinyl |
| A-137 | R¹.38 | vinyl |
| A-138 | R¹.39 | vinyl |
| A-139 | R¹.40 | vinyl |
| A-140 | R¹.41 | vinyl |
| A-141 | R¹.42 | vinyl |
| A-142 | R¹.43 | vinyl |
| A-143 | R¹.44 | vinyl |
| A-144 | R¹.45 | vinyl |
| A-145 | R¹.46 | vinyl |
| A-146 | R¹.47 | vinyl |
| A-147 | R¹.48 | vinyl |
| A-148 | R¹.49 | vinyl |
| A-149 | R¹.40 | vinyl |
| A-150 | R¹.51 | vinyl |
| A-151 | R¹.52 | vinyl |
| A-152 | R¹.53 | vinyl |
| A-153 | R¹.54 | vinyl |
| A-154 | R¹.55 | vinyl |
| A-155 | R¹.56 | vinyl |
| A-156 | R¹.57 | vinyl |
| A-157 | R¹.32 | cyclopropyl |
| A-158 | R¹.33 | cyclopropyl |
| A-159 | R¹.34 | cyclopropyl |
| A-160 | R¹.35 | cyclopropyl |
| A-161 | R¹.36 | cyclopropyl |
| A-162 | R¹.37 | cyclopropyl |
| A-163 | R¹.38 | cyclopropyl |
| A-164 | R¹.39 | cyclopropyl |
| A-165 | R¹.40 | cyclopropyl |
| A-166 | R¹.41 | cyclopropyl |
| A-167 | R¹.42 | cyclopropyl |
| A-168 | R¹.43 | cyclopropyl |
| A-169 | R¹.44 | cyclopropyl |
| A-170 | R¹.45 | cyclopropyl |
| A-171 | R¹.46 | cyclopropyl |
| A-172 | R¹.47 | cyclopropyl |
| A-173 | R¹.48 | cyclopropyl |
| A-174 | R¹.49 | cyclopropyl |
| A-175 | R¹.40 | cyclopropyl |
| A-176 | R¹.51 | cyclopropyl |
| A-177 | R¹.52 | cyclopropyl |
| A-178 | R¹.53 | cyclopropyl |
| A-179 | R¹.54 | cyclopropyl |
| A-180 | R¹.55 | cyclopropyl |
| A-181 | R¹.56 | cyclopropyl |
| A-182 | R¹.57 | cyclopropyl |
| A-183 | R¹.32 | allyl |
| A-184 | R¹.33 | allyl |
| A-185 | R¹.34 | allyl |
| A-186 | R¹.35 | allyl |
| A-187 | R¹.36 | allyl |
| A-188 | R¹.37 | allyl |
| A-189 | R¹.38 | allyl |
| A-190 | R¹.39 | allyl |
| A-191 | R¹.40 | allyl |
| A-192 | R¹.41 | allyl |
| A-193 | R¹.42 | allyl |
| A-194 | R¹.43 | allyl |
| A-195 | R¹.44 | allyl |
| A-196 | R¹.45 | allyl |
| A-197 | R¹.46 | allyl |
| A-198 | R¹.47 | allyl |
| A-199 | R¹.48 | allyl |
| A-200 | R¹.49 | allyl |
| A-201 | R¹.40 | allyl |
| A-202 | R¹.51 | allyl |
| A-203 | R¹.52 | allyl |
| A-204 | R¹.53 | allyl |
| A-205 | R¹.54 | allyl |
| A-206 | R¹.55 | allyl |
| A-207 | R¹.56 | allyl |
| A-208 | R¹.57 | allyl |

The compounds of the formula I can be prepared according to methods or in analogy to methods that are described in the prior art. Compounds, wherein m is 0 and W is #1-(C═O)—NR²-#2 or #1-(C═S)—NR²-#2 can be prepared as described in WO 2015/185485 A1 or in analogy to processes described herein. The synthesis of compounds of the formula I takes advantage of starting materials that are commercially available or may be prepared according to conventional procedures starting from readily available compounds. For example, compounds of the formula I wherein W contains a group —(C═S)— can be prepared in two steps by reacting of an oxadiazole amine, for example compound of the formula II, with compounds of type IIIa (for example acetic chloride or anhydride, LG=chlorine or acetate) in an organic solvent and in the presence of a base followed by thionylation of the obtained carboxamide with either Lawesson's reagent or diphosphorus pentasulfide, as previously described (see, for example, Journal of Organic Chemistry, 2008, 73, 9102 or European Journal of Organic Chemistry, 2015, 30, 6687).

Compounds of formula I wherein W contains a group —S(═O)ₚ— can be prepared by reacting compounds of the formula II in the presence of a base with compounds of the formula IIIb wherein LG is preferably chlorine.

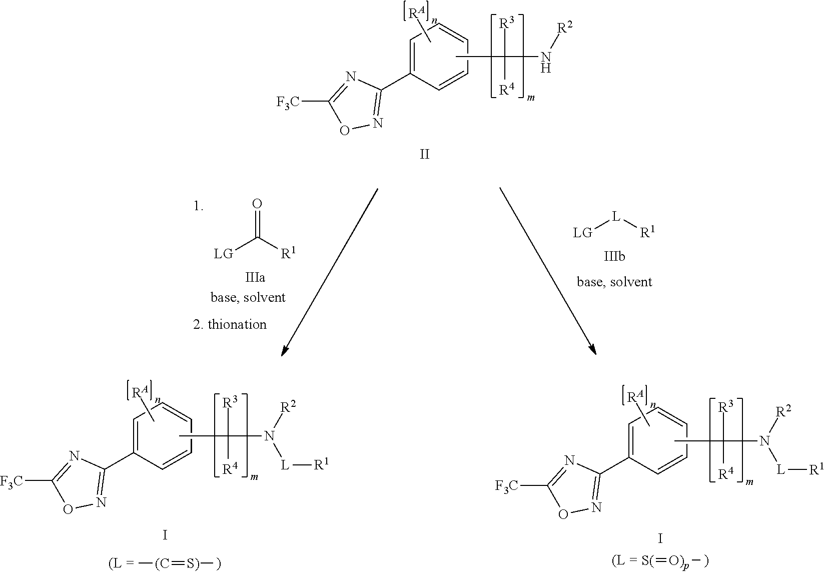

Likewise compounds of the formula I, wherein m is 1 and W is #1-(C═O)—NR²-#2 or #1-(C═S)—NR²-#2 may be prepared as described before, for example in WO 2013008162 from compounds of formula II.a. The process includes activation of the carboxylic acid functionality through conversion into, for example, the carboxylic acid chloride, followed by reaction of the acid chloride with an amine HNR¹R².

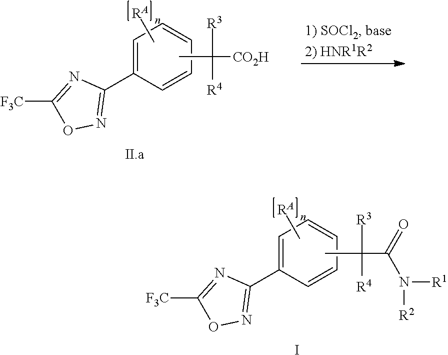

Compounds of the formula II and, in an analogous approach also compounds of the formula II.a, can be prepared by reacting amidoximes of type IV with trifluoroacetic anhydride in an organic solvent, preferably an ethereal solvent at temperatures between 0° C. and 100° C., preferably at room temperature, as previously described in WO 2013/008162 or EP 276432.

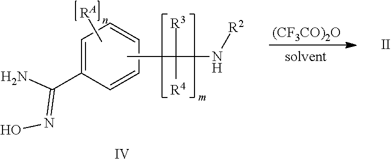

A skilled person will recognize that compounds of type IV can be accessed by treating nitriles of type V with hydroxylamine (or its hydrochloric acid salt) in an organic solvent and in the presence of a base (for precedents see for example WO 2009/074950, WO 2006/013104). Preferably ethanol and potassium carbonate. If appropriate, water may be added to enhance solubility of the reactants. The reaction is best performed at elevated temperatures, most preferably in the range between 60° C. and 80° C.

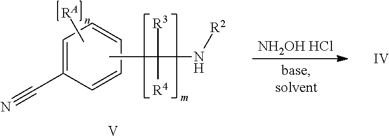

Compounds V are either commercially available or can be accessed from readily available starting materials through methods that are known to a person skilled in the art.

An alternative synthesis route to obtain compounds of formula I starts from a compound of the formula V, which is reacted with compounds IIIa or IIIb as described above. The resulting sulfonamide or carboxamide compounds VI still bears the cyano group, which is subsequently converted into the oxadiazole ring to produce compounds of the formula I via a two step cyclization reaction with hydroxylamine hydrochloride and trifluoroacetic anhydride as described above. The carboxamide may further be thionylated as described above.

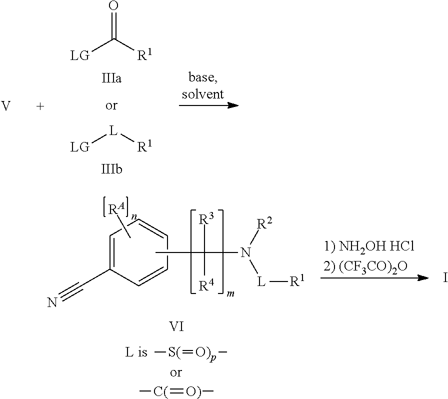

The compounds of the formula I or compositions comprising said compounds according to the invention and the mixtures comprising said compounds and compositions, respectively, are suitable as fungicides. They are distinguished by an outstanding effectiveness against a broad spectrum of phytopathogenic fungi, including soil-borne fungi, which derive especially from the following classes or are closely related to any of them: Ascomycota (Ascomycetes), for example, but not limited to the genus Cocholiobolus, Colletotrichum, Fusarium, Microdochium, Penicillium, Phoma, Magnaporte, Zymoseptoria, and Pseudocercosporella; Basdiomycota (Basidiomycetes), for example, but not limited to the genus Phakospora, Puccinia, Rhizoctonia, Sphacelotheca, Tilletia, Typhula, and Ustilago; Chytridiomycota (Chytridiomycetes), for example, but not limited to the genus Chytridiales, and Synchytrium; Deuteromycetes (syn. Fungi imperfecti), for example, but not limited to the genus Ascochyta, Diplodia, Eysiphe, Fusarium, Phomopsis, and Pyrenophora; Peronosporomycetes (syn. Oomycetes), for example but not limited to the genus Peronospora, Pythium, Phytophthora; Plasmodiophoromycetes, for example but not limited to the genus Plasmodiophora; Zygomycetes, for example, but not limited to the genus Rhizopus.

Some of the compounds of the formula I and the compositions according to the invention are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by mutagenesis or genetic engineering in order to provide a new trait to a plant or to modify an already present trait. Mutagenesis includes techniques of random mutagenesis using X-rays or mutagenic chemicals, but also techniques of targeted mutagenesis, to create mutations at a specific locus of a plant genome. Targeted mutagenesis techniques frequently use oligonucleotides or proteins like CRISPR/Cas, zinc-finger nucleases, TALENs or mega-nucleases to achieve the targeting effect. Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant to add a trait or improve a trait. These integrated genes are also referred to as transgenes in the art, while plant comprising such transgenes are referred to as transgenic plants. The process of plant transformation usually produces several transformation events, wich differ in the genomic locus in which a transgene has been integrated. Plants comprising a specific transgene on a specific genomic locus are usually described as comprising a specific “event”, which is referred to by a specific event name. Traits which have been introduced in plants or have been modified include herbicide tolerance, insect resistance, increased yield and tolerance to abiotic conditions, like drought.

Herbicide tolerance has been created by using mutagenesis as well as using genetic engineering. Plants which have been rendered tolerant to acetolactate synthase (ALS) inhibitor herbicides by mutagenesis and breeding comprise plant varieties commercially available under the name Clearfield®.

Herbicide tolerance has been created via the use of transgenes to glyphosate, glufosinate, 2,4-D, dicamba, oxynil herbicides, like bromoxynil and ioxynil, sulfonylurea herbicides, ALS inhibitors and 4-hydroxyphenylpyruvate dioxygenase (HPPD) inhibitors, like isoxaflutole and mesotrione.

Transgenes which have been used to provide herbicide tolerance traits comprise: for tolerance to glyphosate: cp4 epsps, epsps grg23ace5, mepsps, 2mepsps, gat4601, gat4621, goxv247; for tolerance to glufosinate: pat and bar, for tolerance to 2,4-D: aad-1, aad-12; for tolerance to dicamba: dmo; for tolerance to oxynil herbicies: bxn; for tolerance to sulfonylurea herbicides: zm-hra, csr1-2, gm-hra, S4-HrA; for tolerance to ALS inhibitors: csr1-2; and for tolerance to HPPD inhibitors: hppdPF, W336, avhppd-03.

Transgenic corn events comprising herbicide tolerance genes include, but are not limited to, DAS40278, MON801, MON802, MON809, MON810, MON832, MON87411, MON87419, MON87427, MON88017, MON89034, NK603, GA21, MZHG0JG, HCEM485, VCO-Ø1981-5, 676, 678, 680, 33121, 4114, 59122, 98140, Bt10, Bt176, CBH-351, DBT418, DLL25, MS3, MS6, MZIR098, T25, TC1507 and TC6275.

Transgenic soybean events comprising herbicide tolerance genes include, but are not limited to, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTØH2, W62, W98, FG72 and CV127.

Transgenic cotton events comprising herbicide tolerance genes include, but are not limited to, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40.

Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Insect resistance has mainly been created by transferring bacterial genes for insecticidal proteins to plants: Transgenes which have most frequently been used are toxin genes of Bacillus spp. and synthetic variants thereof, like cry1A, cry1Ab, cry1Ab-Ac, cry1Ac, cry1A.105, cry1F, cry1Fa2, cry2Ab2, cry2Ae, mcry3A, ecry3.1Ab, cry3Bb1, cry34Ab1, cry35Ab1, cry9C, vip3A(a), vip3Aa20. However, also genes of plant origin, such as genes coding for protease inhibitors, like CpTI and pinII, have been transferred to other plants. A further approach uses transgenes such as dvsnf7 to produce double-stranded RNA in plants. Transgenic corn events comprising genes for insecticidal proteins or double stranded RNA include, but are not limited to, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098. Transgenic soybean events comprising genes for insecticidal proteins include, but are not limited to, MON87701, MON87751 and DAS-81419. Transgenic cotton events comprising genes for insecticidal proteins include, but are not limited to, SGK321, MON531, MON757, MON1076, MON15985, 31707, 31803, 31807, 31808, 42317, BNLA-601, Event1, COT67B, COT102, T303-3, T304-40, GFM Cry1A, GK12, MLS 9124, 281-24-236, 3006-210-23, GHB119 and SGK321.

Increased yield has been created by using the transgene athb17, being present for example in corn event MON87403, or by using the transgene bbx32, being present for example in the soybean event MON87712.

Cultivated plants comprising a modified oil content have been created by using the transgenes: gm-fad2-1, Pj.D6D, Nc.Fad3, fad2-1A and fatb1-A. Soybean events comprising at least one of these genes are: 260-05, MON87705 and MON87769.

Tolerance to abiotic conditions, such as drought, has been created by using the transgene cspB, comprised by the corn event MON87460 and by using the transgene Hahb-4, comprised by soybean event IND-ØØ41Ø-5.

Traits are frequently combined by combining genes in a transformation event or by combining different events during the breeding process resulting in a cultivated plant with stacked traits. Preferred combinations of traits are combinations of herbicide tolerance traits to different groups of herbicides, combinations of insect tolerance to different kind of insects, in particular tolerance to lepidopteran and coleopteran insects, combinations of herbicide tolerance with one or several types of insect resistance, combinations of herbicide tolerance with increased yield as well as combinations of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assessment (CERA)” (http://cera-gmc.org/GMCropDatabase). Further information on specific events and methods to detect them can be found for canola events MS1, MS8, RF3, GT73, MON88302, KK179 in WO01/031042, WO01/041558, WO01/041558, WO02/036831, WO11/153186, WO13/003558, for cotton events MON1445, MON15985, MON531 (MON15985), LLCotton25, MON88913, COT102, 281-24-236, 3006-210-23, COT67B, GHB614, T304-40, GHB119, MON88701, 81910 in WO02/034946, WO02/100163, WO02/100163, WO03/013224, WO04/072235, WO04/039986, WO05/103266, WO05/103266, WO06/128573, WO07/017186, WO08/122406, WO08/151780, WO12/134808, WO13/112527; for corn events GA21, MON810, DLL25, TC1507, MON863, MIR604, LY038, MON88017, 3272, 59122, NK603, MIR162, MON89034, 98140, 32138, MON87460, 5307, 4114, MON87427, DAS40278, MON87411, 33121, MON87403, MON87419 in WO98/044140, U.S. Ser. No. 02/102,582, U.S. Ser. No. 03/126,634, WO04/099447, WO04/011601, WO05/103301, WO05/061720, WO05/059103, WO06/098952, WO06/039376, US2007/292854, WO07/142840, WO07/140256, WO08/112019, WO09/103049, WO09/111263, WO10/077816, WO11/084621, WO11/062904, WO11/022469, WO13/169923, WO14/116854, WO15/053998, WO15/142571; for potato events E12, F10, J3, J55, V11, X17, Y9 in WO14/178910, WO14/178913, WO14/178941, WO14/179276, WO16/183445, WO17/062831, WO17/062825; for rice events LLRICE06, LLRICE601, LLRICE62 in WO00/026345, WO00/026356, WO00/026345; and for soybean events H7-1, MON89788, A2704-12, A5547-127, DP305423, DP356043, MON87701, MON87769, CV127, MON87705, DAS68416-4, MON87708, MON87712, SYHT0H2, DAS81419, DAS81419×DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The use of compounds I and compositions according to the invention, respectively, on cultivated plants may result in effects which are specific to a cultivated plant comprising a certain gene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigour, early or delayed ripening, cold or heat tolerance as well as changed amino acid or fatty acid spectrum or content.

The compounds I and compositions thereof, respectively, are particularly suitable for controlling the following plant diseases:

Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Eysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or ‘rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii(orange rust) on sugar cane and P. asparagi on asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni(Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici(Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

In a preferred embodiment the compounds I, their mixtures with other active compounds as defined herein and compositions thereof, respectively, are particularly suitable for controlling the following plant diseases: Puccinia spp. (rusts) on various plants, for example, but not limited to P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, and Puccinia sorghi (common rust) on maize, Puccinia polysora (southern rust) on maize; and Phakopsoraceae spp. on various plants, in particular Phakopsora pachyrhizi and P. meibomiae (soybean rust) on soybeans.

The compounds I and compositions thereof, respectively, are also suitable for controlling harmful fungi in the protection of stored products or harvest and in the protection of materials.

The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold.

Preferably “stored products” is understood to denote natural substances of plant origin and their processed forms, more preferably fruits and their processed forms, such as pomes, stone fruits, soft fruits and citrus fruits and their processed forms.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I according to the invention.

An agrochemical composition comprises a fungicidally effective amount of a compound I.

The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials such as seeds (e. g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; DMSO; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof.

Suitable solid carriers or fillers are mineral earths, e. g. silicates, silica gels, talc, kaolins, limestone, lime, chalk, clays, dolomite, diatomaceous earth, bentonite, calcium sulfate, magnesium sulfate, magnesium oxide; polysaccharides, e. g. cellulose, starch; fertilizers, e. g. ammonium sulfate, ammonium phosphate, ammonium nitrate, ureas; products of vegetable origin, e. g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines.

Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target.

Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives such as alkyliso-thiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

i) Water-soluble concentrates (SL, LS)
10-60 wt % of a compound I and 5-15 wt % wetting agent (e. g. alcohol alkoxylates) are dissolved in water and/or in a water-soluble solvent (e. g. alcohols) ad 100 wt %. The active substance dissolves upon dilution with water.
ii) Dispersible concentrates (DC)
5-25 wt % of a compound I and 1-10 wt % dispersant (e. g. polyvinyl pyrrolidone) are dissolved in organic solvent (e. g. cyclohexanone) ad 100 wt %. Dilution with water gives a dispersion.
iii) Emulsifiable concentrates (EC)
15-70 wt % of a compound I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

### iv) Emulsions (EW, EO, ES)

5-40 wt % of a compound I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

### v) Suspensions (SC, OD, FS)

In an agitated ball mill, 20-60 wt % of a compound I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension. Dilution with water gives a stable suspension of the active substance. For FS type composition up to 40 wt % binder (e. g. polyvinyl alcohol) is added.
vi) Water-dispersible granules and water-soluble granules (WG, SG)
50-80 wt % of a compound I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.
vii) Water-dispersible powders and water-soluble powders (WP, SP, WS)
50-80 wt % of a compound I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.
viii) Gel (GW, GF)
In an agitated ball mill, 5-25 wt % of a compound I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

### ix) Microemulsion (ME)

5-20 wt % of a compound I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

### x) Microcapsules (CS)

An oil phase comprising 5-50 wt % of a compound I, 0-40 wt % water insoluble organic solvent (e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound I according to the invention, 0-40 wt % water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.
xi) Dustable powders (DP, DS)
1-10 wt % of a compound I are ground finely and mixed intimately with solid carrier (e. g. finely divided kaolin) ad 100 wt %.
xii) Granules (GR, FG)
0.5-30 wt % of a compound I is ground finely and associated with solid carrier (e. g. silicate) ad 100 wt %. Granulation is achieved by extrusion, spray-drying or fluidized bed.
xiii) Ultra-low volume liquids (UL)
1-50 wt % of a compound I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, more preferably between 1 and 70%, and in particular between 10 and 60%, by weight of active substance. The active substances are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed.

The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, onto plant propagation material, especially seeds, include dressing, coating, pelleting, dusting, and soaking as well as in-furrow application methods. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

A pesticide is generally a chemical or biological agent (such as pestidal active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease.

The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of pesticides II (e. g. pesticidally-active substances and biopesticides), in conjunction with which the compounds I can be used, is intended to illustrate the possible combinations but does not limit them:

A) Respiration inhibitors
Inhibitors of complex III at Qₒ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxy-strobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylidene-aminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21a), methyl-N-[2-[(1,4-dimethyl-5-phenyl-pyrazol-3-yl)oxylmethyl]phenyl]-N-methoxy-carbamate (A.1.22), metyltetrapole (A.1.25), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethyl-pent-3-enamide (A1.35), pyriminostrobin (A.1.36), bifujunzhi (A.1.37), 2-(ortho-((2,5-dimeth-ylphenyl-oxymethylen)phenyl)-3-methoxy-acrylic acid methylester (A.1.38);
inhibitors of complex III at Qᵢ site: cyazofamid (A.2.1), amisulbrom (A.2.2), [(6S,7R,8R)-8-benzyl-3-[(3-hydroxy-4-methoxy-pyridine-2-carbonyl)amino]-6-methyl-4,9-dioxo-1,5-dioxonan-7-yl] 2-methylpropanoate (A2.3), fenpicoxamid (A.2.4), florylpicoxamid (A.2.5);
inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), pyraziflumid (A.3.18), sedaxane (A.3.19), tecloftalam (A.3.20), thifluzamide (A.3.21), inpyrfluxam (A.3.22), pyrapropoyne (A.3.23), fluindapyr (A.3.28), methyl (E)-2-[2-[(5-cyano-2-methyl-phenoxy)methyl]phenyl]-3-methoxy-prop-2-enoate (A.3.30), isoflucypram (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]pyridine-3-carboxamide (A.3.33), 2-(difluoromethyl)-N-(3-ethyl-1,1-dimethyl-indan-4-yl)pyridine-3-carboxamide (A.3.34), 2-(difluoromethyl)-N-[(3R)-3-ethyl-1,1-dimethyl-indan-4-yl]pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]-pyridine-3-carboxamide (A.3.37), 2-(difluoromethyl)-N-(3-isobutyl-1,1-dimethyl-indan-4-yl)pyridine-3-carboxamide (A.3.38), 2-(difluoromethyl)-N-[(3R)-3-isobutyl-1,1-dimethyl-indan-4-yl]pyridine-3-carboxamide (A.3.39);
other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);
   B) Sterol biosynthesis inhibitors (SBI fungicides)
C14 demethylase inhibitors: triazoles: azaconazole (B.1.1), bitertanol (B.1.2), bromu-conazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothio-conazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 2-(2,4-difluorophenyl)-1,1-difluoro-3-(tetrazol-1-yl)-1-[5-[4-(2,2,2-trifluoroethoxy)phenyl]-2-pyridyl]propan-2-ol (B.1.31), 2-(2,4-difluorophenyl)-1,1-difluoro-3-(tetrazol-1-yl)-1-[5-[4-(trifluoromethoxy)phenyl]-2-pyridyl]propan-2-ol (B.1.32), ipfentrifluconazole (B.1.37), mefentrifluconazole (B.1.38), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines, piperazines: fenarimol (B.1.49), pyrifenox (B.1.50), triforine (B.1.51), [3-(4-chloro-2-fluoro-phenyl)-5-(2,4-difluorophenyl)isoxazol-4-yl]-(3-pyridyl)methanol (B.1.52);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);
Other Sterol biosynthesis inhibitors: chlorphenomizole (B.4.1);
   C) Nucleic acid synthesis inhibitors
phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);
   D) Inhibitors of cell division and cytoskeleton
tubulin inhibitors: benomyl (D.1.1), carbendazim (D.1.2), fuberidazole (D1.3), thiabendazole (D.1.4), thiophanate-methyl (D.1.5), pyridachlometyl (D.1.6), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-flu-oroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluoro-phenyl)-N-(2-chloro-6-fluoro-phenyl)-2,5-dimethyl-pyrazol-3-amine (D.1.16);
other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7);
   E) Inhibitors of amino acid and protein synthesis
methionine synthesis inhibitors: cyprodinil (E.1.1), mepanipyrim (E.1.2), pyrimethanil (E.1.3);
protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6);
   F) Signal transduction inhibitors
MAP/histidine kinase inhibitors: fluoroimid (F.1.1), iprodione (F.1.2), procymidone (F.1.3), vinclozolin (F.1.4), fludioxonil (F.1.5);
G protein inhibitors: quinoxyfen (F.2.1);
   G) Lipid and membrane synthesis inhibitors
Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);
compounds affecting cell membrane permeability and fatty acides: propamocarb (G.4.1);
inhibitors of oxysterol binding protein: oxathiapiprolin (G.5.1), 2-{3-[2-(1-{[3,5-bis(difluoro-methyl-1H-pyrazol-1-yl]acetyl}piperidin-4-yl)-1,3-thiazol-4-yl]-4,5-dihydro-1,2-oxazol-5-yl}-phenyl methanesulfonate (G.5.2), 2-{3-[2-(1-{[3,5-bis(difluoromethyl)-1H-pyrazol-1-yl]-acetyl}piperidin-4-yl) 1,3-thiazol-4-yl]-4,5-dihydro-1,2-oxazol-5-yl}-3-chlorophenyl methanesulfonate (G.5.3), 4-[1-[2-[3-(difluoromethyl)-5-methyl-pyrazol-1-yl]acetyl]-4-piperidyl]-N-tetralin-1-yl-pyridine-2-carboxamide (G.5.4), 4-[1-[2-[3,5-bis(difluoromethyl)pyrazol-1-yl]acetyl]-4-piperidyl]-N-tetralin-1-yl-pyridine-2-carboxamide (G.5.5), 4-[1-[2-[3-(difluoromethyl)-5-(trifluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl)pyrazol-1-yl]acetyl]-4-piperidyl]-N-tetralin-1-yl-pyridine-2-carboxamide (G.5.7), 4-[1-[2-[5-methyl-3-(trifluoromethyl)pyrazol-1-yl]acetyl]-4-piperidyl]-N-tetralin-1-yl-pyridine-2-carboxamide (G.5.8), 4-[1-[2-[5-(difluoromethyl)-3-(trifluoromethyl)pyrazol-1-yl]acetyl]-4-piperidyl]-N-tetralin-1-yl-pyridine-2-carboxamide (G.5.9), 4-[1-[2-[3,5-bis(trifluoromethyl)pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11);
   H) Inhibitors with Multi Site Action
inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachloro-benzene (H.3.8), pentachlorphenole (H.3.9) and its salts, phthalide (H.3.10), tolylfluanid (H.3.11);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);
   I) Cell wall synthesis inhibitors
inhibitors of glucan synthesis: validamycin (I.1.1), polyoxin B (I.1.2);
melanin synthesis inhibitors: pyroquilon (I.2.1), tricyclazole (I.2.2), carpropamid (I.2.3), dicyclomet (I.2.4), fenoxanil (I.2.5);
   J) Plant defence inducers
acibenzolar-S-methyl (J.1.1), probenazole (J.1.2), isotianil (J.1.3), tiadinil (J.1.4), prohexa-dione-calcium (J.1.5); phosphonates: fosetyl (J.1.6), fosetyl-aluminum (J.1.7), phosphorous acid and its salts (J.1.8), calcium phosphonate (J.1.11), potassium phosphonate (J.1.12), potassium or sodium bicarbonate (J.1.9), 4-cyclopropyl-N-(2,4-di-methoxyphenyl)thiadiazole-5-carboxamide (J.1.10);
   K) Unknown mode of action
bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitropan (K.1.12), fenpyrazamine (K.1.13), flumetover (K.1.14), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxin-copper (K.1.22), proquinazid (K.1.23), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methyl formamidine (K.1.28), N′-[4-[[3-[(4-chlorophenyl)methyl]-1,2,4-thiadiazol-5-yl]oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl-3-pyridyl]-N-ethyl-N-methyl-formamidine (K.1.31), N′-[5-bromo-6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsilanyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrimidin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), ipflufenoquin (K.1.44), quinofumelin (K.1.47), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), pyrifenamine (K.1.54);
   M) Growth regulators
   abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid, trinexapac-ethyl, uniconazole;
   N) Herbicides from classes N.1 to N.15
N.1 Lipid biosynthesis inhibitors: alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-chloro-4-cyclo¬propyl-2′-fluoro[1,1′-biphenyl]-3-yl)-5-hydroxy-2,2,6,6-tetramethyl-2H-pyran-3(6H)-one (1312337-72-6); 4-(2′,4′-dichloro-4-cyclopropyl[1,1′-biphenyl]-3-yl)-5-hydroxy-2,2,6,6-tetramethyl-2H-pyran-3(6H)-one (1312337-45-3); 4-(4′-chloro-4-ethyl-2′-fluoro[1,1′-biphenyl]-3-yl)-5-hydroxy-2,2,6,6-tetramethyl-2H-pyran-3(6H)-one (1033757-93-5); 4-(2′,4′-dichloro-4-ethyl[1,1′-biphenyl]-3-yl)-2,2,6,6-tetramethyl-2H-pyran-3,5(4H,6H)-dione (1312340-84-3); 5-(acetyloxy)-4-(4′-chloro-4-cyclopropyl-2′-fluoro[1,1′-biphenyl]-3-yl)-3,6-dihydro-2,2,6,6-tetramethyl-2H-pyran-3-one (1312337-48-6); 5-(acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(acetyloxy)-4-(4′-chloro-4-ethyl-2′-fluoro[1,1′-biphenyl]-3-yl)-3,6-dihydro-2,2,6,6-tetramethyl-2H-pyran-3-one (1312340-82-1); 5-(acetyloxy)-4-(2′,4′-dichloro-4-ethyl[1,1′-biphenyl]-3-yl)-3,6-dihydro-2,2,6,6-tetramethyl-2H-pyran-3-one (1033760-55-2); 4-(4′-chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (1312337-51-1); 4-(2′,4′-dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (1312340-83-2); 4-(2′,4′-dichloro-4-ethyl¬[1,1′-biphenyl]-3-yl)-5,6-dihydro-2,2,6,6-tetramethyl-5-oxo-2H-pyran-3-yl carbonic acid methyl ester (1033760-58-5); benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate, vernolate;
N.2 ALS inhibitors: amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, iodosulfuron, iodosulfuron-methyl-sodium, iofensulfuron, iofensul-furon-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl, tritosulfuron, imazamethabenz, imazamethabenz-methyl, imazamox, imazapic, imazapyr, imazaquin, imazethapyr; cloransulam, cloransulam-methyl, diclosulam, flumetsulam, florasulam, metosulam, penoxsulam, pyrimisulfan, pyroxsulam; bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methyl¬ethyl ester (420138-41-6), 4-[[[2-[(4,6-dimethoxy-2-pyrimidinyl)oxy]phenyl]¬methyl]amino]-benzoic acid propyl ester (420138-40-5), N-(4-bromophenyl)-2-[(4,6-dimethoxy-2-pyrimidinyl)oxy]benzenemethanamine (420138-01-8); flucarbazone, flucarbazone-sodium, propoxycarbazone, propoxycarbazone-sodium, thiencarbazone, thiencarbazone-methyl; triafamone;
N.3 Photosynthesis inhibitors: amicarbazone; chlorotriazine; ametryn, atrazine, chloridazone, cyanazine, desmetryn, dimethametryn, hexazinone, metribuzin, prometon, prometryn, pro-pazine, simazine, simetryn, terbumeton, terbuthylazin, terbutryn, trietazin; chlorobrom-uron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron, thiadiazuron, desmedipham, karbutilat, phenmedipham, phenmedipham-ethyl, bromofenoxim, bromoxynil and its salts and esters, ioxynil and its salts and esters, bromacil, lenacil, terbacil, bentazon, bentazon-sodium, pyridate, pyridafol, pentanochlor, propanil; diquat, diquat-dibromide, paraquat, paraquat-dichloride, paraquat-dimetilsulfate;
N.4 protoporphyrinogen-IX oxidase inhibitors: acifluorfen, acifluorfen-sodium, azafenidin, bencarbazone, benzfendizone, bifenox, butafenacil, carfentrazone, carfentrazone-ethyl, chlormethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (353292-31-6), N-ethyl-3-(2,6-dichloro-4-trifluoro-methylphenoxy)-5-methyl-1H-pyrazole-1-carboxamide (452098-92-9), N-tetrahydrofurfuryl-3-(2,6-dichloro-4-trifluoromethylphenoxy)-5-methyl-1H-pyrazole-1-carboxamide (915396-43-9), N-ethyl-3-(2-chloro-6-fluoro-4-trifluoromethyl¬phenoxy)-5-methyl-1H-pyrazole-1-carboxamide (452099-05-7), N-tetrahydro¬furfuryl-3-(2-chloro-6-fluoro-4-trifluoro¬methylphenoxy)-5-methyl-1H-pyrazole-1-carboxamide (452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (1300118-96-0), 1-methyl-6-trifluoro¬methyl-3-(2,2,7-tri-fluoro-3-oxo-4-prop-2-ynyl-3,4-dihydro-2H-benzo[1,4]oxazin-6-yl)-1H-pyrimidine-2,4-dione (1304113-05-0), methyl (E)-4-[2-chloro-5-[4-chloro-5-(difluoromethoxy)-1H-methyl-pyrazol-3-yl]-4-fluoro-phenoxy]-3-methoxy-but-2-enoate (948893-00-3), 3-[7-chloro-5-fluoro-2-(trifluoromethyl)-1H-benzimidazol-4-yl]-1-methyl-6-(trifluoromethyl)-1H-pyrimidine-2,4-dione (212754-02-4);
N.5 Bleacher herbicides: beflubutamid, diflufenican, fluridone, flurochloridone, flurtamone, norflurazon, picolinafen, 4-(3-trifluoromethyl¬phenoxy)-2-(4-trifluoromethylphenyl)pyrimidine (180608-33-7); benzobicyclon, benzofenap, bicyclopyrone, clomazone, fenquintrione, isoxaflutole, mesotrione, pyrasulfotole, pyrazolynate, pyrazoxyfen, sulcotrione, tefuryltrione, tembotrione, tolpyralate, topramezone; aclonifen, amitrole, flumeturon;
N.6 EPSP synthase inhibitors: glyphosate, glyphosate-isopropylammonium, glyposate-potassium, glyphosate-trimesium (sulfosate);
N.7 Glutamine synthase inhibitors: bilanaphos (bialaphos), bilanaphos-sodium, glufosinate, glufosinate-P, glufosinate-ammonium;
N.8 DHP synthase inhibitors: asulam;
N.9 Mitosis inhibitors: benfluralin, butralin, dinitramine, ethalfluralin, fluchloralin, oryzalin, pendimethalin, prodiamine, trifluralin; amiprophos, amiprophos-methyl, butamiphos; chlorthal, chlorthal-dimethyl, dithiopyr, thiazopyr, propyzamide, tebutam; carbetamide, chlorpropham, flamprop, flamprop-isopropyl, flamprop-methyl, flamprop-M-isopropyl, flamprop-M-methyl, propham;
N.10 VLCFA inhibitors: acetochlor, alachlor, butachlor, dimethachlor, dimethenamid, dimethenamid-P, metazachlor, metolachlor, metolachlor-S, pethoxamid, pretilachlor, propachlor, propisochlor, thenylchlor, flufenacet, mefenacet, diphenamid, naproanilide, napropamide, napropamide-M, fentrazamide, anilofos, cafenstrole, fenoxasulfone, ipfencarbazone, piperophos, pyroxasulfone, isoxazoline compounds of the formulae II.1, II.2, II.3, II.4, II.5, II.6, II.7, II.8 and II.9

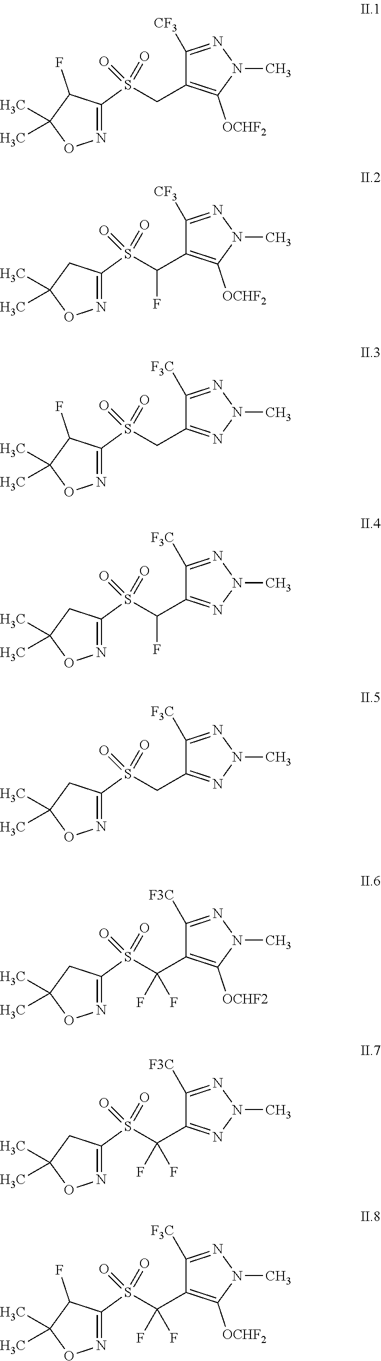

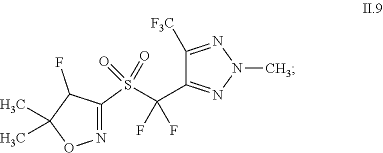 [SMILES: Cn1nc(C(F)(F)F)c(C(F)(F)S(=O)(=O)C2=NOC(C)(C)C2F)n1]

N.11 Cellulose biosynthesis inhibitors: chlorthiamid, dichlobenil, flupoxam, indaziflam, isoxaben, triaziflam, 1-cyclohexyl-5-pentafluorphenyloxy-14-[1,2,4,6]thiatriazin-3-ylamine (175899-01-1);
N.12 Decoupler herbicides: dinoseb, dinoterb, DNOC and its salts;
N.13 Auxinic herbicides: 2,4-D and its salts and esters, clacyfos, 2,4-DB and its salts and esters, aminocyclopyrachlor and its salts and esters, aminopyralid and its salts such as aminopyralid-dimethylammonium, aminopyralid-tris(2-hydroxypropyl)ammonium and its esters, benazolin, benazolin-ethyl, chloramben and its salts and esters, clomeprop, clopy-ralid and its salts and esters, dicamba and its salts and esters, dichlorprop and its salts and esters, dichlorprop-P and its salts and esters, fluroxypyr, fluroxypyr-butometyl, fluroxypyr-meptyl, halauxifen and its salts and esters (943832-60-8); MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, 4-amino-3-chloro-6-(4-chloro-2-fluoro-3-methoxyphenyl)-5-fluoropyridine-2-carboxylic acid, benzyl 4-amino-3-chloro-6-(4-chloro-2-fluoro-3-methoxyphenyl)-5-fluoropyridine-2-carboxylate (1390661-72-9);
N.14 Auxin transport inhibitors: diflufenzopyr, diflufenzopyr-sodium, naptalam, naptalam-sodium;
N.15 Other herbicides: bromobutide, chlorflurenol, chlorflurenol-methyl, cinmethylin, cumyluron, cyclopyrimorate (499223-49-3) and its salts and esters, dalapon, dazomet, difenzoquat, difenzoquat-metilsulfate, dimethipin, DSMA, dymron, endothal and its salts, etobenzanid, flurenol, flurenol-butyl, flurprimidol, fosamine, fosamine-ammonium, indanofan, maleic hydrazide, mefluidide, metam, methiozolin (403640-27-7), methyl azide, methyl bromide, methyl-dymron, methyl iodide, MSMA, oleic acid, oxaziclomefone, pelargonic acid, pyributicarb, quinoclamine, tridiphane;
   O) Insecticides from classes O.1 to O.29
O.1 Acetylcholine esterase (AChE) inhibitors: aldicarb (O.1.1), alanycarb (O.1.2), bendiocarb (O.1.3), benfuracarb (O.1.4), butocarboxim (O.1.5), butoxycarboxim (O.1.6), carbaryl (O.1.7), carbofuran (O.1.8), carbosulfan (O.1.9), ethiofencarb (O.1.10), fenobucarb (O.1.11), formetanate (O.1.12), furathiocarb (O.1.13), isoprocarb (O.1.14), methiocarb (O.1.15), methomyl (O.1.16), metolcarb (O.1.17), oxamyl (O.1.18), pirimicarb (O.1.19), propoxur (O.1.20), thiodicarb (O.1.21), thiofanox (O.1.22), trimethacarb (O.1.23), XMC (O.1.24), xylylcarb (O.1.25), triazamate (O.1.26), acephate (O.1.27), azamethiphos (O.1.28), azinphos-ethyl (O.1.29), azinphosmethyl (O.1.30), cadusafos (O.1.31), chlorethoxyfos (O.1.32), chlorfenvinphos (O.1.33), chlormephos (O.1.34), chlorpyrifos (O.1.35), chlorpyrifos-methyl (O.1.36), coumaphos (O.1.37), cyanophos (O.1.38), demeton-S-methyl (O.1.39), diazinon (O.1.40), dichlorvos/DDVP (O.1.41), dicrotophos (O.1.42), dimethoate (O.1.43), dimethylvinphos (O.1.44), disulfoton (O.1.45), EPN (O.1.46), ethion (O.1.47), ethoprophos (O.1.48), famphur (O.1.49), fenamiphos (O.1.50), fenitrothion (O.1.51), fenthion (O.1.52), fosthiazate (O.1.53), heptenophos (O.1.54), imicyafos (O.1.55), isofenphos (O.1.56), isopropyl 0-(methoxyaminothio-phosphoryl) salicylate (O.1.57), isoxathion (O.1.58), malathion (O.1.59), mecarbam (O.1.60), methamidophos (O.1.61), methidathion (O.1.62), mevinphos (O.1.63), monocrotophos (O.1.64), naled (O.1.65), omethoate (O.1.66), oxydemeton-methyl (O.1.67), parathion (O.1.68), parathion-methyl (O.1.69), phenthoate (O.1.70), phorate (O.1.71), phosalone (O.1.72), phosmet (O.1.73), phosphamidon (O.1.74), phoxim (O.1.75), pirimiphos-methyl (O.1.76), profenofos (O.1.77), propetamphos (O.1.78), prothiofos (O.1.79), pyraclofos (O.1.80), pyridaphenthion (O.1.81), quinalphos (O.1.82), sulfotep (O.1.83), tebupirimfos (O.1.84), temephos (O.1.85), terbufos (O.1.86), tetrachlorvinphos (O.1.87), thiometon (O.1.88), triazophos (O.1.89), trichlorfon (O.1.90), vamidothion (O.1.91);
O.2 GABA-gated chloride channel antagonists: endosulfan (O.2.1), chlordane (O.2.2), ethiprole (O.2.3), fipronil (O.2.4), flufiprole (O.2.5), pyrafluprole (O.2.6), pyriprole (O.2.7);
O.3 Sodium channel modulators: acrinathrin (O.3.1), allethrin (O.3.2), d-cis-trans allethrin (O.3.3), d-trans allethrin (O.3.4), bifenthrin (O.3.5), kappa-bifenthrin (O.3.6), bioallethrin (O.3.7), bioallethrin S-cylclopentenyl (O.3.8), bioresmethrin (O.3.9), cycloprothrin (O.3.10), cyfluthrin (O.3.11), beta-cyfluthrin (O.3.12), cyhalothrin (O.3.13), lambda-cyhalothrin (O.3.14), gamma-cyhalothrin (O.3.15), cypermethrin (O.3.16), alpha-cypermethrin (O.3.17), beta-cypermethrin (O.3.18), theta-cypermethrin (O.3.19), zeta-cypermethrin (O.3.20), cyphenothrin (O.3.21), deltamethrin (O.3.22), empenthrin (O.3.23), esfenvalerate (O.3.24), etofenprox (O.3.25), fenpropathrin (O.3.26), fenvalerate (O.3.27), flucythrinate (O.3.28), flumethrin (O.3.29), tau-fluvalinate (O.3.30), halfenprox (O.3.31), heptafluthrin (O.3.32), imiprothrin (O.3.33), meperfluthrin (O.3.34), metofluthrin (O.3.35), momfluorothrin (O.3.36), epsilon-momfluorothrin (O.3.37), permethrin (O.3.38), phenothrin (O.3.39), prallethrin (O.3.40), profluthrin (O.3.41), pyrethrin (pyrethrum) (O.3.42), resmethrin (O.3.43), silafluofen (O.3.44), tefluthrin (O.3.45), kappa-tefluthrin (O.3.46), tetramethylfluthrin (O.3.47), tetramethrin (O.3.48), tralomethrin (O.3.49), transfluthrin (O.3.50), DDT (O.3.51), methoxychlor (O.3.52);
O.4 Nicotinic acetylcholine receptor agonists (nAChR): acetamiprid (O.4.1), clothianidin (O.4.2), cycloxaprid (O.4.3), dinotefuran (O.4.4), imidacloprid (O.4.5), nitenpyram (O.4.6), thiacloprid (O.4.7), thiamethoxam (O.4.8), 4,5-dihydro-N-nitro-1-(2-oxiranylmethyl)-1H-imidazol-2-amine (O.4.9), (2E)-1-[(6-chloropyridin-3-yl)methyl]-N′-nitro-2-pentylidene-hydrazinecarboximidamide (O.4.10), 1-[(6-chloropyridin-3-yl)methyl]-7-methyl-8-nitro-5-propoxy-1,2,3,5,6,7-hexahydroimidazo[1,2-a]pyridine (O.4.11), nicotine (O.4.12), sulfoxaflor (O.4.13), flupyradifurone (O.4.14), triflumezopyrim (O.4.15);
O.5 Nicotinic acetylcholine receptor allosteric activators: spinosad (O.5.1), spinetoram (O.5.2);
O.6 Chloride channel activators: abamectin (O.6.1), emamectin benzoate (O.6.2), ivermectin (O.6.3), lepimectin (O.6.4), milbemectin (O.6.5);
O.7 Juvenile hormone mimics: hydroprene (O.7.1), kinoprene (O.7.2), methoprene (O.7.3), fenoxycarb (O.7.4), pyriproxyfen (O.7.5);
O.8 miscellaneous non-specific (multi-site) inhibitors: methyl bromide (O.8.1) and other alkyl halides, chloropicrin (O.8.2), sulfuryl fluoride (O.8.3), borax (O.8.4), tartar emetic (O.8.5);
O.9 Chordotonal organ TRPV channel modulators: pymetrozine (O.9.1), pyrifluquinazon (O.9.2), flonicamid (O.9.3);

O.10 Mite growth inhibitors: clofentezine (O.10.1), hexythiazox (O.10.2), diflovidazin (O.10.3), etoxazole (O.10.4);
O.11 Microbial disruptors of insect midgut membranes: Bacillus thuringiensis, Bacillus sphaericus and the insecticidal proteins they produce: Bacillus thuringiensis subsp. Israelensis (O.11.1), Bacillus sphaericus (O.11.2), Bacillus thuringiensis subsp. aizawai (O.11.3), Bacillus thuringiensis subsp. kurstaki (O.11.4), Bacillus thuringiensis subsp. tenebrionis (O.11.5), the Bt crop proteins: Cry1Ab (O.11.6), Cry1Ac (O.11.7), Cry1Fa (O.11.8), Cry2Ab (O.11.9), mCry3A (O.11.10), Cry3Ab (O.11.11), Cry3Bb (O.11.12), Cry34/35Ab1 (O.11.13);
O.12 Inhibitors of mitochondrial ATP synthase: diafenthiuron (O.12.1), azocyclotin (O.12.2), cyhexatin (O.12.3), fenbutatin oxide (O.12.4), propargite (O.12.5), tetradifon (O.12.6);
O.13 Uncouplers of oxidative phosphorylation via disruption of the proton gradient: chlorfenapyr (O.13.1), DNOC (O.13.2), sulfluramid (O.13.3);
O.14 Nicotinic acetylcholine receptor (nAChR) channel blockers: bensultap (O.14.1), cartap hydrochloride (O.14.2), thiocyclam (O.14.3), thiosultap sodium (O.14.4);
O.15 Inhibitors of the chitin biosynthesis type 0: bistrifluron (O.15.1), chlorfluazuron (O.15.2), diflubenzuron (O.15.3), flucycloxuron (O.15.4), flufenoxuron (O.15.5), hexaflumuron (O.15.6), lufenuron (O.15.7), novaluron (O.15.8), noviflumuron (O.15.9), teflubenzuron (O.15.10), triflumuron (O.15.11);
O.16 Inhibitors of the chitin biosynthesis type 1: buprofezin (O.16.1);
O.17 Moulting disruptors: cyromazine (O.17.1);
O.18 Ecdyson receptor agonists: methoxyfenozide (O.18.1), tebufenozide (O.18.2), halofenozide (O.18.3), fufenozide (O.18.4), chromafenozide (O.18.5);
O.19 Octopamin receptor agonists: amitraz (O.19.1);
O.20 Mitochondrial complex III electron transport inhibitors: hydramethylnon (O.20.1), acequinocyl (O.20.2), fluacrypyrim (O.20.3), bifenazate (O.20.4);
O.21 Mitochondrial complex I electron transport inhibitors: fenazaquin (O.21.1), fenpyroximate (O.21.2), pyrimidifen (O.21.3), pyridaben (O.21.4), tebufenpyrad (O.21.5), tolfenpyrad (O.21.6), rotenone (O.21.7);
O.22 Voltage-dependent sodium channel blockers: indoxacarb (O.22.1), metaflumizonev (O.22.2), 2-[2-(4-cyanophenyl)-1-[3-(trifluoromethyl)phenyl]ethylidene]-N-[4-(difluoromethoxy)phenyl]-hydrazinecarboxamide (O.22.3), N-(3-chloro-2-methylphenyl)-2-[(4-chlorophenyl)-[4-[methyl(methylsulfonyl)amino]phenyl]methylene]-hydrazinecarboxamide (O.22.4);
O.23 Inhibitors of the of acetyl CoA carboxylase: spirodiclofen (O.23.1), spiromesifen (O.23.2), spirotetramat (O.23.3), spiropidion (O.23.4);
O.24 Mitochondrial complex IV electron transport inhibitors: aluminium phosphide (O.24.1), calcium phosphide (O.24.2), phosphine (O.24.3), zinc phosphide (O.24.4), cyanide (O.24.5);
O.25 Mitochondrial complex II electron transport inhibitors: cyenopyrafen (O.25.1), cyflumetofen (O.25.2);
O.26 Ryanodine receptor-modulators: flubendiamide (O.26.1), chlorantraniliprole (O.26.2), cyantraniliprole (O.26.3), cyclaniliprole (O.26.4), tetraniliprole (O.26.5), (R)-3-chloro-N¹-{2-methyl-4-[1,2,2,2-tetrafluoro-1-(trifluoromethyl)ethyl]phenyl}-N²-(1-methyl-2-methylsulfonylethyl)phthalamide (O.26.6), (S)-3-chloro-N¹-{2-methyl-4-[1,2,2,2-tetrafluoro-1-(trifluoromethyl)ethyl]phenyl}-N²-(1-methyl-2-methylsulfonylethyl)phthalamide (O.26.7), methyl-2-[3,5-dibromo-2-({[3-bromo-1-(3-chloropyridin-2-yl)-1H-pyrazol-5-yl]-carbonyl}amino)benzoyl]-1,2-dimethylhydrazinecarboxylate (O.26.8), N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide (O.26.9), N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide (O.26.10), N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide (O.26.11), N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide (O.26.12), N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide (O.26.13), N-[2-(5-amino-1,3,4-thiadiazol-2-yl)-4-chloro-6-methylphenyl]-3-bromo-1-(3-chloro-2-pyridinyl)-1H-pyrazole-5-carboxamide (O.26.14), 3-chloro-1-(3-chloro-2-pyridinyl)-N-[2,4-dichloro-6-[[(1-cyano-1-methylethyl)amino]carbonyl]phenyl]-1H-pyrazole-5-carboxamide (O.26.15), 3-bromo-N-[2,4-dichloro-6-(methylcarbamoyl)phenyl]-1-(3,5-dichloro-2-pyridyl)-1H-pyrazole-5-carboxamide (O.26.16), N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (O.26.17), cyhalodiamide (O.26.18);
O.27: Chordotonal organ Modulators—undefined target site: flonicamid (O.27.1);
O.28. insecticidal active compounds of unknown or uncertain mode of action: afidopyropen (O.28.1), afoxolaner (O.28.2), azadirachtin (O.28.3), amidoflumet (O.28.4), benzoximate (O.28.5), broflanilide (O.28.6), bromopropylate (O.28.7), chinomethionat (O.28.8), cryolite (O.28.9), dicloromezotiaz (O.28.10), dicofol (O.28.11), flufenerim (O.28.12), flometoquin (O.28.13), fluensulfone (O.28.14), fluhexafon (O.28.15), fluopyram (O.28.16), fluralaner (O.28.17), metoxadiazone (O.28.18), piperonyl butoxide (O.28.19), pyflubumide (O.28.20), pyridalyl (O.28.21), tioxazafen (O.28.22), 11-(4-chloro-2,6-dimethylphenyl)-12-hydroxy-1,4-dioxa-9-azadispiro[4.2.4.2]-tetradec-11-en-10-one, 3-(4′-fluoro-2,4-dimethylbiphenyl-3-yl)-4-hydroxy-8-oxa-1-azaspiro[4.5]dec-3-en-2-one, 1-[2-fluoro-4-methyl-5-[(2,2,2-trifluoroethyl)sulfinyl]phenyl]-3-(trifluoromethyl)-1H-1,2,4-triazole-5-amine (O.28.23), Bacillus firmusI-1582 (O.28.24), flupyrimin (O.28.25), fluazaindolizine (O.28.26), 4-[5-(3,5-dichlorophenyl)-5-(trifluoromethyl)-4H-isoxazol-3-yl]-2-methyl-N-(1-oxothietan-3-yl)benzamide (O.28.27), fluxametamide (O.28.28), 5-[3-[2,6-dichloro-4-(3,3-dichloroallyloxy)phenoxy]propoxy]-1H-pyrazole (O.28.1), 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide (O.28.29), 4-cyano-3-[(4-cyano-2-methyl-benzoyl)amino]-N-[2,6-di-chloro-4-[1,2,2,3,3,3-hexafluoro-1-(trifluoromethyl)propyl]phenyl]-2-fluoro-benzamide (O.28.30), N-[5-[[2-chloro-6-cyano-4-[1,2,2,3,3,3-hexafluoro-1-(trifluoromethyl)propyl]-phenyl]carbamoyl]-2-cyano-phenyl]-4-cyano-2-methyl-benzamide (O.28.31), N-[5[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide (O.28.32), N-[5[[2-bromo-6-chloro-4-[1,2,2,3,3,3-hexafluoro-1-(trifluoromethyl)propyl]phenyl]carbamoyl]-2-cyano-phenyl]-4-cyano-2-methyl-benzamide (O.28.33), 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide (O.28.34), 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide (O.28.35),

N-[5[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide (O.28.36); 2-(1,3-dioxan-2-yl)-6-[2-(3-pyridinyl)-5-thiazolyl]-pyridine (O.28.37), 2-[6-[2-(5-fluoro-3-pyridinyl)-5-thiazolyl]-2-pyridinyl]-pyrimidine (O.28.38), 2-[6-[2-(3-pyridinyl)-5-thiazolyl]-2-pyridinyl]-pyrimidine (O.28.39), N-methylsulfonyl-6-[2-(3-pyridyl)thiazol-5-yl]pyridine-2-carboxamide (O.28.40), N-methylsulfonyl-6-[2-(3-pyridyl)thiazol-5-yl]pyridine-2-carboxamide (O.28.41), 1-[(6-chlo-ro-3-pyridinyl)methyl]-1,2,3,5,6,7-hexahydro-5-methoxy-7-methyl-8-nitro-imidazo[1,2-a]pyridine (O.28.42), 1-[(6-chloropyridin-3-yl)methyl]-7-methyl-8-nitro-1,2,3,5,6,7-hexahydroimidazo[1,2-a]pyridin-5-ol (O.28.43), 1-isopropyl-N,5-dimethyl-N-pyridazin-4-yl-pyrazole-4-carboxamide (O.28.44), 1-(1,2-dimethylpropyl)-N-ethyl-5-methyl-N-pyridazin-4-yl-pyrazole-4-carboxamide (O.28.45), N,5-dimethyl-N-pyridazin-4-yl-1-(2,2,2-trifluoro-1-methyl-ethyl)pyrazole-4-carboxamide (O.28.46), 1-[1-(1-cyanocyclopropyl)ethyl]-N-ethyl-5-methyl-N-pyridazin-4-yl-pyrazole-4-carboxamide (O.28.47), N-ethyl-1-(2-fluoro-1-methyl-propyl)-5-meth-yl-N-pyridazin-4-yl-pyrazole-4-carboxamide (O.28.48), 1-(1,2-dimethylpropyl)-N,5-dimethyl-N-pyridazin-4-yl-pyrazole-4-carboxamide (O.28.49), 1-[1-(1-cyanocyclopropyl)ethyl]-N,5-dimethyl-N-pyridazin-4-yl-pyrazole-4-carboxamide (O.28.50), N-methyl-1-(2-fluoro-1-methyl-propyl]-5-methyl-N-pyridazin-4-yl-pyrazole-4-carboxamide (O.28.51), 1-(4,4-difluorocyclohexyl)-N-ethyl-5-methyl-N-pyridazin-4-yl-pyr-azole-4-carboxamide (O.28.52), 1-(4,4-difluorocyclohexyl)-N,5-dimethyl-N-pyridazin-4-yl-pyrazole-4-carboxamide (O.28.53), N-(1-methylethyl)-2-(3-pyridinyl)-2H-indazole-4-carboxamide (O.28.54), N-cyclopropyl-2-(3-pyridinyl)-2H-indazole-4-carboxamide (O.28.55), N-cyclohexyl-2-(3-pyridinyl)-2H-indazole-4-carboxamide (O.28.56), 2-(3-pyridinyl)-N-(2,2,2-trifluoroethyl)-2H-indazole-4-carboxamide (O.28.57), 2-(3-pyridinyl)-N-[(tetrahydro-2-furanyl)methyl]-2H-indazole-5-carboxamide (O.28.58), methyl 2-[[2-(3-pyridinyl)-2H-indazol-5-yl]carbonyl]hydrazinecarboxylate (O.28.59), N-[(2,2-di-fluorocyclopropyl)methyl]-2-(3-pyridinyl)-2H-indazole-5-carboxamide (O.28.60), N-(2,2-di-fluoropropyl)-2-(3-pyridinyl)-2H-indazole-5-carboxamide (O.28.61), 2-(3-pyridinyl)-N-(2-pyrimidinylmethyl)-2H-indazole-5-carboxamide (O.28.62), N-[(5-methyl-2-pyrazinyl)-methyl]-2-(3-pyridinyl)-2H-indazole-5-carboxamide (O.28.63), tyclopyrazoflor (O.28.64), sarolaner (O.28.65), lotilaner (O.28.66), N-[4-chloro-3-[[(phenylmethyl)amino]carbonyl]phenyl]-1-methyl-3-(1,1,2,2,2-pentafluoroethyl)-4-(trifluoromethyl)-1H-pyrazole-5-carboxamide (O.28.67), M.UN.22a 2-(3-ethylsulfonyl-2-pyridyl)-3-methyl-6-(trifluoromethyl)imidazo[4,5-b]pyridine (O.28.68), 2-[3-ethylsulfonyl-5-(trifluoromethyl)-2-pyridyl]-3-methyl-6-(trifluoromethyl)imidazo[4,5-b]pyridine (O.28.69), 4-[5-(3,5-dichlorophenyl)-5-(trifluoromethyl)-4H-isoxazol-3-yl]-N—[(R)-2-ethyl-3-oxo-isoxazolidin-4-yl]-2-methyl-benzamide (O.28.70), 4-[5-(3,5-dichloro-4-fluoro-phenyl)-5-(trifluoromethyl)-4H-isoxazol-3-yl]-N—[(R)-2-ethyl-3-oxo-isoxazolidin-4-yl]-2-methyl-benzamide (O.28.71), N-[4-chloro-3-(cyclopropylcarbamoyl)phenyl]-2-methyl-5-(1,1,2,2,2-pentafluoroethyl)-4-(trifluoromethyl)pyrazole-3-carboxamide (O.28.72), N-[4-chloro-3-[(1-cyanocyclopropyl)-carbamoyl]phenyl]-2-methyl-5-(1,1,2,2,2-pentafluoroethyl)-4-(trifluoromethyl)pyrazole-3-carboxamide (O.28.73), acynonapyr (O.28.74), benzpyrimoxan (O.28.75), chloro-N-(1-cyanocyclopropyl)-5-[1-[2-methyl-5-(1,1,2,2,2-pentafluoroethyl)-4-(trifluoromethyl)pyrazol-3-yl]pyrazol-4-yl]benzamide (O.28.76), oxazosulfyl (O.28.77), [(2S,3R,4R,5S,6S)-3,5-dimethoxy-6-methyl-4-propoxy-tetrahydropyran-2-yl]-N-[4-[1-[4-(trifluoromethoxy)phenyl]-1,2,4-triazol-3-yl]phenyl]carbamate (O.28.78), [(2S,3R,4R,5S,6S)-3,4,5-trimethoxy-6-methyl-tetrahydropyran-2-yl] N-[4-[1-[4-(trifluoromethoxy)phenyl]-1,2,4-triazol-3-yl]phenyl]-carbamate (O.28.79), [(2S,3R,4R,5S,6S)-3,5-dimethoxy-6-methyl-4-propoxy-tetrahydropyran-2-yl]-N-[4-[1-[4-(1,1,2,2,2-pentafluoroethoxy)phenyl]-1,2,4-triazol-3-yl]phenyl]carbamate (O.28.80), [(2S,3R,4R,5S,6S)-3,4,5-trimethoxy-6-methyl-tetra-hydropyran-2-yl]-N-[4-[1-[4-(1,1,2,2,2-pentafluoroethoxy)phenyl]-1,2,4-triazol-3-yl]phenyl]-carbamate (O.28.81), (2Z)-3-(2-isopropylphenyl)-2-[(E)-[4-[1-[4-(1,1,2,2,2-pentafluoroethoxy)phenyl]-1,2,4-triazol-3-yl]phenyl]methylenehydrazono]thiazolidin-4-one (O.28.82).

The active substances referred to as component 2, their preparation and their activity e. g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441).

The present invention furthermore relates to agrochemical compositions comprising a mixture of at least one compound I (component 1) and at least one further active substance useful for plant protection, e. g. selected from the groups A) to O) (component 2), in particular one further fungicide, e. g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to K), as described above, is more efficient than combating those fungi with individual compounds I or individual fungicides from groups A) to K).

By applying compounds I together with at least one active substance from groups A) to O) a synergistic effect can be obtained, i.e. more then simple addition of the individual effects is obtained (synergistic mixtures).

This can be obtained by applying the compounds I and at least one further active substance simultaneously, either jointly (e. g. as tank-mix) or seperately, or in succession, wherein the time interval between the individual applications is selected to ensure that the active substance applied first still occurs at the site of action in a sufficient amount at the time of application of the further active substance(s). The order of application is not essential for working of the present invention.

When applying compound I and a pesticide II sequentially the time between both applications may vary e. g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day.

In the binary mixtures and compositions according to the invention the weight ratio of the component 1) and the component 2) generally depends from the properties of the active components used, usually it is in the range of from 1:10,000 to 10,000:1, often it is in the range of from 1:100 to 100:1, regularly in the range of from 1:50 to 50:1, preferably in the range of from 1:20 to 20:1, more preferably in the range of from 1:10 to 10:1, even more preferably in the range of from 1:4 to 4:1 and in particular in the range of from 1:2 to 2:1. According to further embodiments of the binary mixtures and compositions, the weight ratio of the component 1) and the component 2) usually is in the range of from 1000:1 to 1:1, often in the range of from 100:1 to 1:1, regularly in the range of from 50:1 to 1:1, preferably in the range of from 20:1 to 1:1, more preferably in the range of from 10:1 to 1:1, even more preferably in the range of from 4:1 to 1:1 and in particular in the range of from 2:1 to 1:1. According to a further embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:10, even more preferably in the range of from 1:1 to 1:4 and in particular in the range of from 1:1 to 1:2. In the ternary mixtures, i.e. compositions according to the invention comprising the component 1) and component 2) and a compound III (component 3), the weight ratio of component 1) and component 2) depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1, and the weight ratio of component 1) and component 3) usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1.

Any further active components are, if desired, added in a ratio of from 20:1 to 1:20 to the component 1).

These ratios are also suitable for inventive mixtures applied by seed treatment.

Accordingly, the present invention furthermore relates to mixtures comprising one compound of the formula I (component 1, a group represented by the expression “(I)”) and one pesticide II (component 2), wherein pesticide II is an active ingredients selected from the groups A) to O) defined above.

Further embodiments B-1 to B-683 listed in Table B below relate to mixtures comprising as active components one of the in the present specification individualized compounds of the formula I, which is selected from the group of compounds I.A.A-1 to I.A.A-208, I.B.A-1 to I.B.A-208, I.C.A-1 to I.C.A-208, I.D.A-1 to I.D.A-208, I.E.A-1 to I.E.A-208, I.F.A-1 to I.F.A-208, I.G.A-1 to I.G.A-208, I.H.A-1 to I.H.A-208, I.J.A-1 to I.J.A-208 and I.K.A-1 to I.K.A-208, I.L.A-1 to I.L.A-208, I.M.A-1 to I.M.A-208, I.N.A-1 to I.N.A-208, I.O.A-1 to I.O.A-208, I.P.A-1 to I.P.A-208, I.Q.A-1 to I.Q.A-208, I.R.A-1 to I.R.A-208, I.S.A-1 to I.S.A-208, I.T.A-1 to I.T.A-208, I.U.A-1 to I.U.A-208, I.V.A-1 to I.V.A-208, I.W.A-1 to I.W.A-208, I.X.A-1 to I.X.A-208 and I.Y.A-1 to I.Y.A-208 as defined in tables 1 to 24 (component 1, a group represented by the expression “(I)”) and one pesticide II selected from the groups A) to 0) as defined herein (component 2, for example, (A.1.1) or azoxystrobin, in embodiment B-1).

Further embodiments B-1 to B-683 listed in Table B below relate to the mixtures comprising as active components one of the in the present specification individualized compounds of the formula I, which is selected from the group of compounds Ex-1 to Ex-5 as defined in Table I below.

Preferably, the compositions described in Table B comprise the active components in synergistically effective amounts.

### Table B:

B-1: (I)+(A.1.1), B-2: (I)+(A.1.2), B-3: (I)+(A.1.3), B-4: (I)+(A.1.4), B-5: (I)+(A.1.5), B-6: (I)+(A.1.6), B-7: (I)+(A.1.7), B-8: (I)+(A.1.8), B-9: (I)+(A.1.9), B-10: (I)+(A.1.10), B-11: (I)+(A.1.11), B-12: (I)+(A.1.12), B-13: (I)+(A.1.13), B-14: (I)+(A.1.14), B-15: (I)+(A.1.15), B-16: (I)+(A.1.16), B-17: (I)+(A.1.17), B-18: (I)+(A.1.18), B-19: (I)+(A.1.19), B-20: (I)+(A.1.20), B-21: (I)+(A.1.21), B-22: (I)+(A.1.21a), B-23: (I)+(A.1.22), B-24: (I)+(A.1.25), B-25: (I)+(A.1.34), B-26: (I)+(A.1.35), B-27: (I)+(A.1.36), B-28: (I)+(A.1.37), B-29: (I)+(A.1.38), B-30: (I)+(A.2.1), B-31: (I)+(A.2.2), B-32: (I)+(A.2.3), B-33: (I)+(A.2.4), B-34: (I)+(A.2.5), B-35: (I)+(A.3.1), B-36: (I)+(A.3.2), B-37: (I)+(A.3.3), B-38: (I)+(A.3.4), B-39: (I)+(A.3.5), B-40: (I)+(A.3.6), B-41: (I)+(A.3.7), B-42: (I)+(A.3.8), B-43: (I)+(A.3.9), B-44: (I)+(A.3.10), B-45: (I)+(A.3.11), B-46: (I)+(A.3.12), B-47: (I)+(A.3.13), B-48: (I)+(A.3.14), B-49: (I)+(A.3.15), B-50: (I)+(A.3.16), B-51: (I)+(A.3.17), B-52: (I)+(A.3.18), B-53: (I)+(A.3.19), B-54: (I)+(A.3.20), B-55: (I)+(A.3.21), B-56: (I)+(A.3.22), B-57: (I)+(A.3.23), B-58: (I)+(A.3.24), B-59: (I)+(A.3.25), B-60: (I)+(A.3.26), B-61: (I)+(A.3.27), B-62: (I)+(A.3.28), B-63: (I)+(A.3.30), B-64: (I)+(A.3.31), B-65: (I)+(A.3.32), B-66: (I)+(A.3.33), B-67: (I)+(A.3.34), B-68: (I)+(A.3.35), B-69: (I)+(A.3.36), B-70: (I)+(A.3.37), B-71: (I)+(A.3.38), B-72: (I)+(A.3.39), B-73: (I)+(A.4.1), B-74: (I)+(A.4.2), B-75: (I)+(A.4.3), B-76: (I)+(A.4.4), B-77: (I)+(A.4.5), B-78: (I)+(A.4.6), B-79: (I)+(A.4.7), B-80: (I)+(A.4.8), B-81: (I)+(A.4.9), B-82: (I)+(A.4.10), B-83: (I)+(A.4.11), B-84: (I)+(A.4.12), B-85: (I)+(B.1.1), B-86: (I)+(B.1.2), B-87: (I)+(B.1.3), B-88: (I)+(B.1.4), B-89: (I)+(B.1.5), B-90: (I)+(B.1.6), B-91: (I)+(B.1.7), B-92: (I)+(B.1.8), B-93: (I)+(B.1.9), B-94: (I)+(B.1.10), B-95: (I)+(B.1.11), B-96: (I)+(B.1.12), B-97: (I)+(B.1.13), B-98: (I)+(B.1.14), B-99: (I)+(B.1.15), B-100: (I)+(B.1.16), B-101: (I)+(B.1.17), B-102: (I)+(B.1.18), B-103: (I)+(B.1.19), B-104: (I)+(B.1.20), B-105: (I)+(B.1.21), B-106: (I)+(B.1.22), B-107: (I)+(B.1.23), B-108: (I)+(B.1.24), B-109: (I)+(B.1.25), B-110: (I)+(B.1.26), B-111: (I)+(B.1.27), B-112: (I)+(B.1.28), B-113: (I)+(B.1.29), B-114: (I)+(B.1.30), B-115: (I)+(B.1.31), B-116: (I)+(B.1.32), B-117: (I)+(B.1.37), B-118: (I)+(B.1.38), B-119: (I)+(B.1.39), B-120: (I)+(B.1.40), B-121: (I)+(B.1.41), B-122: (I)+(B.1.42), B-123: (I)+(B.1.43), B-124: (I)+(B.1.44), B-125: (I)+(B.1.45), B-126: (I)+(B.1.46), B-127: (I)+(B.1.47), B-128: (I)+(B.1.48), B-129: (I)+(B.1.49), B-130: (I)+(B.1.50), B-131: (I)+(B.1.51), B-132: (I)+(B.1.52), B-133: (I)+(B.2.1), B-134: (I)+(B.2.2), B-135: (I)+(B.2.3), B-136: (I)+(B.2.4), B-137: (I)+(B.2.5), B-138: (I)+(B.2.6), B-139: (I)+(B.2.7), B-140: (I)+(B.2.8), B-141: (I)+(B.3.1), B-142: (I)+(B.4.1), B-143: (I)+(C.1.1), B-144: (I)+(C.1.2), B-145: (I)+(C.1.3), B-146: (I)+(C.1.4), B-147: (I)+(C.1.5), B-148: (I)+(C.1.6), B-149: (I)+(C.1.7), B-150: (I)+(C.2.1), B-151: (I)+(C.2.2), B-152: (I)+(C.2.3), B-153: (I)+(C.2.4), B-154: (I)+(C.2.5), B-155: (I)+(C.2.6), B-156: (I)+(C.2.7), B-157: (I)+(C.2.8), B-158: (I)+(D.1.1), B-159: (I)+(D.1.2), B-160: (I)+(D.1.3), B-161: (I)+(D.1.4), B-162: (I)+(D.1.5), B-163: (I)+(D.1.6), B-164: (I)+(D.1.7), B-165: (I)+(D.1.8), B-166: (I)+(D.1.9), B-167: (I)+(D.1.10), B-168: (I)+(D.1.11), B-169: (I)+(D.1.12), B-170: (I)+(D.1.13), B-171: (I)+(D.1.14), B-172: (I)+(D.1.15), B-173: (I)+(D.1.16), B-174: (I)+(D.2.1), B-175: (I)+(D.2.2), B-176: (I)+(D.2.3), B-177: (I)+(D.2.4), B-178: (I)+(D.2.5), B-179: (I)+(D.2.6), B-180: (I)+(D.2.7), B-181: (I)+(E.1.1), B-182: (I)+(E.1.2), B-183: (I)+(E.1.3), B-184: (I)+(E.2.1), B-185: (I)+(E.2.2), B-186: (I)+(E.2.3), B-187: (I)+(E.2.4), B-188: (I)+(E.2.5), B-189: (I)+(E.2.6), B-190: (I)+(F.1.1), B-191: (I)+(F.1.2), B-192: (I)+(F.1.3), B-193: (I)+(F.1.4), B-194: (I)+(F.1.5), B-195: (I)+(F.2.1), B-196: (I)+(G.1.1), B-197: (I)+(G.1.2), B-198: (I)+(G.1.3), B-199: (I)+(G.1.4), B-200: (I)+(G.2.1), B-201: (I)+(G.2.2), B-202: (I)+(G.2.3), B-203: (I)+(G.2.4), B-204: (I)+(G.2.5), B-205: (I)+(G.2.6), B-206: (I)+(G.2.7), B-207: (I)+(G.3.1), B-208: (I)+(G.3.2), B-209: (I)+(G.3.3), B-210: (I)+(G.3.4), B-211: (I)+(G.3.5), B-212: (I)+(G.3.6), B-213: (I)+(G.3.7), B-214: (I)+(G.4.1), B-215: (I)+(G.5.1), B-216: (I)+(G.5.2), B-217: (I)+(G.5.3), B-218: (I)+(G.5.4), B-219: (I)+(G.5.5), B-220: (I)+(G.5.6), B-221: (I)+(G.5.7), B-222: (I)+(G.5.8), B-223: (I)+(G.5.9), B-224: (I)+(G.5.10), B-225: (I)+(G.5.11), B-226: (I)+(H.1.1), B-227: (I)+(H.1.2), B-228: (I)+(H.1.3), B-229: (I)+(H.1.4), B-230: (I)+(H.1.5), B-231: (I)+(H.1.6), B-232: (I)+(H.1.7), B-233: (I)+(H.2.1), B-234: (I)+(H.2.2), B-235: (I)+(H.2.3), B-236: (I)+(H.2.4), B-237: (I)+(H.2.5), B-238: (I)+(H.2.6), B-239: (I)+(H.2.7), B-240: (I)+(H.2.8), B-241: (I)+(H.2.9), B-242: (I)+(H.3.1), B-243: (I)+(H.3.2), B-244: (I)+(H.3.3), B-245: (I)+(H.3.4), B-246: (I)+(H.3.5), B-247: (I)+(H.3.6), B-248: (I)+(H.3.7), B-249: (I)+(H.3.8), B-250: (I)+(H.3.9), B-251: (I)+(H.3.10), B-252: (I)+(H.3.11), B-253: (I)+(H.4.1), B-254: (I)+(H.4.2), B-255: (I)+(H.4.3), B-256: (I)+(H.4.4), B-257: (I)+(H.4.5), B-258: (I)+(H.4.6), B-259: (I)+(H.4.7), B-260: (I)+(H.4.8), B-261: (I)+(H.4.9), B-262: (I)+(H.4.10), B-263: (I)+(1.1.1), B-264: (I)+(1.1.2), B-265: (I)+(1.2.1), B-266: (I)+(1.2.2), B-267: (I)+(1.2.3), B-268: (I)+(1.2.4), B-269: (I)+(1.2.5), B-270: (I)+(J.1.1), B-271: (I)+(J.1.2), B-272: (I)+(J.1.3), B-273: (I)+(J.1.4), B-274: (I)+(J.1.5), B-275: (I)+(J.1.6), B-276: (I)+(J.1.7), B-277: (I)+(J.1.8), B-278: (I)+(J.1.9), B-279: (I)+(J.1.10), B-280: (I)+(K.1.1), B-281: (I)+(K.1.2), B-282: (I)+(K.1.3), B-283: (I)+(K.1.4), B-284: (I)+(K.1.5), B-285: (I)+(K.1.6), B-286: (I)+(K.1.7), B-287: (I)+(K.1.8), B-288: (I)+(K.1.9), B-289: (I)+(K.1.10), B-290: (I)+(K.1.11), B-291: (I)+(K.1.12), B-292: (I)+(K.1.13), B-293: (I)+(K.1.14), B-294: (I)+(K.1.15), B-295: (I)+(K.1.16), B-296: (I)+(K.1.17), B-297: (I)+(K.1.18), B-298: (I)+(K.1.19), B-299: (I)+(K.1.20), B-300: (I)+(K.1.21), B-301: (I)+(K.1.22), B-302: (I)+(K.1.23), B-303: (I)+(K.1.24), B-304: (I)+(K.1.25), B-305: (I)+(K.1.26), B-306: (I)+(K.1.27), B-307: (I)+(K.1.28), B-308: (I)+(K.1.29), B-309: (I)+(K.1.30), B-310: (I)+(K.1.31), B-311: (I)+(K.1.32), B-312: (I)+(K.1.33), B-313: (I)+(K.1.34), B-314: (I)+(K.1.35), B-315: (I)+(K.1.36), B-316: (I)+(K.1.37), B-317: (I)+(K.1.38), B-318: (I)+(K.1.39), B-319: (I)+(K.1.40), B-320: (I)+(K.1.41), B-321: (I)+(K.1.42), B-322: (I)+(K.1.43), B-323: (I)+(K.1.44), B-324: (I)+(K.1.45), B-325: (I)+(K.1.46), B-326: (I)+(K.1.47), B-327: (I)+(K.1.48), B-328: (I)+(K.1.49), B-329: (I)+(K.1.50), B-330: (I)+(K.1.51), B-331: (I)+(K.1.52), B-332: (I)+(K.1.53), B-333: (I)+(K.1.54), B-334: (I)+(O.1.1), B-335: (I)+(O.1.2), B-336: (I)+(O.1.3), B-337: (I)+(O.1.4), B-338: (I)+(O.1.5), B-339: (I)+(O.1.6), B-340: (I)+(O.1.7), B-341: (I)+(O.1.8), B-342: (I)+(O.1.9), B-343: (I)+(O.1.10), B-344: (I)+(O.1.11), B-345: (I)+(O.1.12), B-346: (I)+(O.1.13), B-347: (I)+(O.1.14), B-348: (I)+(O.1.15), B-349: (I)+(O.1.16), B-350: (I)+(O.1.17), B-351: (I)+(O.1.18), B-352: (I)+(O.1.19), B-353: (I)+(O.1.20), B-354: (I)+(O.1.21), B-355: (I)+(O.1.22), B-356: (I)+(O.1.23), B-357: (I)+(O.1.24), B-358: (I)+(O.1.25), B-359: (I)+(O.1.26), B-360: (I)+(O.1.27), B-361: (I)+(O.1.28), B-362: (I)+(O.1.29), B-363: (I)+(O.1.30), B-364: (I)+(O.1.31), B-365: (I)+(O.1.32), B-366: (I)+(O.1.33), B-367: (I)+(O.1.34), B-368: (I)+(O.1.35), B-369: (I)+(O.1.36), B-370: (I)+(O.1.37), B-371: (I)+(O.1.38), B-372: (I)+(O.1.39), B-373: (I)+(O.1.40), B-374: (I)+(O.1.41), B-375: (I)+(O.1.42), B-376: (I)+(O.1.43), B-377: (I)+(O.1.44), B-378: (I)+(O.1.45), B-379: (I)+(O.1.46), B-380: (I)+(O.1.47), B-381: (I)+(O.1.48), B-382: (I)+(O.1.49), B-383: (I)+(O.1.50), B-384: (I)+(O.1.51), B-385: (I)+(O.1.52), B-386: (I)+(O.1.53), B-387: (I)+(O.1.54), B-388: (I)+(O.1.55), B-389: (I)+(O.1.56), B-390: (I)+(O.1.57), B-391: (I)+(O.1.58), B-392: (I)+(O.1.59), B-393: (I)+(O.1.60), B-394: (I)+(O.1.61), B-395: (I)+(O.1.62), B-396: (I)+(O.1.63), B-397: (I)+(O.1.64), B-398: (I)+(O.1.65), B-399: (I)+(O.1.66), B-400: (I)+(O.1.67), B-401: (I)+(O.1.68), B-402: (I)+(O.1.69), B-403: (I)+(O.1.70), B-404: (I)+(O.1.71), B-405: (I)+(O.1.72), B-406: (I)+(O.1.73), B-407: (I)+(O.1.74), B-408: (I)+(O.1.75), B-409: (I)+(O.1.76), B-410: (I)+(O.1.77), B-411: (I)+(O.1.78), B-412: (I)+(O.1.79), B-413: (I)+(O.1.80), B-414: (I)+(O.1.81), B-415: (I)+(O.1.82), B-416: (I)+(O.1.83), B-417: (I)+(O.1.84), B-418: (I)+(O.1.85), B-419: (I)+(O.1.86), B-420: (I)+(O.1.87), B-421: (I)+(O.1.88), B-422: (I)+(O.1.89), B-423: (I)+(O.1.90), B-424: (I)+(O.1.91), B-425: (I)+(O.2.1), B-426: (I)+(O.2.2), B-427: (I)+(O.2.3), B-428: (I)+(O.2.4), B-429: (I)+(O.2.5), B-430: (I)+(O.2.6), B-431: (I)+(O.2.7), B-432: (I)+(O.3.1), B-433: (I)+(O.3.2), B-434: (I)+(O.3.3), B-435: (I)+(O.3.4), B-436: (I)+(O.3.5), B-437: (I)+(O.3.6), B-438: (I)+(O.3.7), B-439: (I)+(O.3.8), B-440: (I)+(O.3.9), B-441: (I)+(O.3.10), B-442: (I)+(O.3.11), B-443: (I)+(O.3.12), B-444: (I)+(O.3.13), B-445: (I)+(O.3.14), B-446: (I)+(O.3.15), B-447: (I)+(O.3.16), B-448: (I)+(O.3.17), B-449: (I)+(O.3.18), B-450: (I)+(O.3.19), B-451: (I)+(O.3.20), B-452: (I)+(O.3.21), B-453: (I)+(O.3.22), B-454: (I)+(O.3.23), B-455: (I)+(O.3.24), B-456: (I)+(O.3.25), B-457: (I)+(O.3.26), B-458: (I)+(O.3.27), B-459: (I)+(O.3.28), B-460: (I)+(O.3.29), B-461: (I)+(O.3.30), B-462: (I)+(O.3.31), B-463: (I)+(O.3.32), B-464: (I)+(O.3.33), B-465: (I)+(O.3.34), B-466: (I)+(O.3.35), B-467: (I)+(O.3.36), B-468: (I)+(O.3.37), B-469: (I)+(O.3.38), B-470: (I)+(O.3.39), B-471: (I)+(O.3.40), B-472: (I)+(O.3.41), B-473: (I)+(O.3.42), B-474: (I)+(O.3.43), B-475: (I)+(O.3.44), B-476: (I)+(O.3.45), B-477: (I)+(O.3.46), B-478: (I)+(O.3.47), B-479: (I)+(O.3.48), B-480: (I)+(O.3.49), B-481: (I)+(O.3.50), B-482: (I)+(O.3.51), B-483: (I)+(O.3.52), B-484: (I)+(O.4.1), B-485: (I)+(O.4.2), B-486: (I)+(O.4.3), B-487: (I)+(O.4.4), B-488: (I)+(O.4.5), B-489: (I)+(O.4.6), B-490: (I)+(O.4.7), B-491: (I)+(O.4.8), B-492: (I)+(O.4.9), B-493: (I)+(O.4.10), B-494: (I)+(O.4.11), B-495: (I)+(O.4.12), B-496: (I)+(O.4.13), B-497: (I)+(O.4.14), B-498: (I)+(O.4.15), B-499: (I)+(O.5.1), B-500: (I)+(O.5.2), B-501: (I)+(O.6.1), B-502: (I)+(O.6.2), B-503: (I)+(O.6.3), B-504: (I)+(O.6.4), B-505: (I)+(O.6.5), B-506: (I)+(O.7.1), B-507: (I)+(O.7.2), B-508: (I)+(O.7.3), B-509: (I)+(O.7.4), B-510: (I)+(O.7.5), B-511: (I)+(O.8.1), B-512: (I)+(O.8.2), B-513: (I)+(O.8.3), B-514: (I)+(O.8.4), B-515: (I)+(O.8.5), B-516: (I)+(O.9.1), B-517: (I)+(O.9.2), B-518: (I)+(O.9.3), B-519: (I)+(O.10.1), B-520: (I)+(O.10.2), B-521: (I)+(O.10.3), B-522: (I)+(O.10.4), B-523: (I)+(O.11.1), B-524: (I)+(O.11.2), B-525: (I)+(O.11.3), B-526: (I)+(O.11.4), B-527: (I)+(O.11.5), B-528: (I)+(O.11.6), B-529: (I)+(O.11.7), B-530: (I)+(O.11.8), B-531: (I)+(O.11.9), B-532: (I)+(O.11.10), B-533: (I)+(O.11.11), B-534: (I)+(O.11.12), B-535: (I)+(O.11.13), B-536: (I)+(O.12.1), B-537: (I)+(O.12.2), B-538: (I)+(O.12.3), B-539: (I)+(O.12.4), B-540: (I)+(O.12.5), B-541: (I)+(O.12.6), B-542: (I)+(O.13.1), B-543: (I)+(O.13.2), B-544: (I)+(O.13.3), B-545: (I)+(O.14.1), B-546: (I)+(O.14.2), B-547: (I)+(O.14.3), B-548: (I)+(O.14.4), B-549: (I)+(O.15.1), B-550: (I)+(O.15.2), B-551: (I)+(O.15.3), B-552: (I)+(O.15.4), B-553: (I)+(O.15.5), B-554: (I)+(O.15.6), B-555: (I)+(O.15.7), B-556: (I)+(O.15.8), B-557: (I)+(O.15.9), B-558: (I)+(O.15.10), B-559: (I)+(O.15.11), B-560: (I)+(O.16.1), B-561: (I)+(O.17.1), B-562: (I)+(O.18.1), B-563: (I)+(O.18.2), B-564: (I)+(O.18.3), B-565: (I)+(O.18.4), B-566: (I)+(O.18.5), B-567: (I)+(O.19.1), B-568: (I)+(O.20.1), B-569: (I)+(O.20.2), B-570: (I)+(O.20.3), B-571: (I)+(O.20.4), B-572: (I)+(O.21.1), B-573: (I)+(O.21.2), B-574: (I)+(O.21.3), B-575: (I)+(O.21.4), B-576: (I)+(O.21.5), B-577: (I)+(O.21.6), B-578: (I)+(O.21.7), B-579: (I)+(O.22.1), B-580: (I)+(O.22.2), B-581: (I)+(O.22.3), B-582: (I)+(O.22.4), B-583: (I)+(O.23.1), B-584: (I)+(O.23.2), B-585: (I)+(O.23.3), B-586: (I)+(O.23.4), B-587: (I)+(O.24.1), B-588: (I)+(O.24.2), B-589: (I)+(O.24.3), B-590: (I)+(O.24.4), B-591: (I)+(O.24.5), B-592: (I)+(O.25.1), B-593: (I)+(O.25.2), B-594: (I)+(O.26.1), B-595: (I)+(O.26.2), B-596: (I)+(O.26.3), B-597: (I)+(O.26.4), B-598: (I)+(O.26.5), B-599: (I)+(O.26.6), B-600: (I)+(O.26.7), B-601: (I)+(O.26.8), B-602: (I)+(O.26.9), B-603: (I)+(O.26.10), B-604: (I)+(O.26.11), B-605: (I)+(O.26.12), B-606: (I)+(O.26.13), B-607: (I)+(O.26.14), B-608: (I)+(O.26.15), B-609: (I)+(O.26.16), B-610: (I)+(O.26.17), B-611: (I)+(O.26.18), B-612: (I)+(O.27.1), B-613: (I)+(O.28.1), B-614: (I)+(O.28.2), B-615: (I)+(O.28.3), B-616: (I)+(O.28.4), B-617: (I)+(O.28.5), B-618: (I)+(O.28.7), B-619: (I)+(O.28.8), B-620: (I)+(O.28.9), B-621: (I)+(O.28.10), B-622: (I)+(O.28.11), B-623: (I)+(O.28.12), B-624: (I)+(O.28.13), B-625: (I)+(O.28.14), B-626: (I)+(O.28.15), B-627: (I)+(O.28.16), B-628: (I)+(O.28.17), B-629: (I)+(O.28.18), B-630: (I)+(O.28.19), B-631: (I)+(O.28.20), B-632: (I)+(O.28.21), B-633: (I)+(O.28.22), B-634: (I)+(O.28.23), B-635: (I)+(O.28.24), B-636: (I)+(O.28.25), B-637: (I)+(O.28.26), B-638: (I)+(O.28.27), B-639: (I)+(O.28.28), B-640: (I)+(O.28.29), B-641: (I)+(O.28.30), B-642: (I)+(O.28.31), B-643: (I)+(O.28.42), B-644: (I)+(O.28.43), B-645: (I)+(O.28.44), B-646: (I)+(O.28.45), B-647: (I)+(O.28.46), B-648: (I)+(O.28.47), B-649: (I)+(O.28.48), B-650: (I)+(O.28.49), B-651: (I)+(O.28.50), B-652: (I)+(O.28.51), B-653: (I)+(O.28.52), B-654: (I)+(O.28.53), B-655: (I)+(O.28.54), B-656: (I)+(O.28.55), B-657: (I)+(O.28.56), B-658: (I)+(O.28.57), B-659: (I)+(O.28.58), B-660: (I)+(O.28.59), B-661: (I)+(O.28.60), B-662: (I)+(O.28.61), B-663: (I)+(O.28.62), B-664: (I)+(O.28.63), B-665: (I)+(O.28.64), B-666: (I)+(O.28.65), B-667: (I)+(O.28.66), B-668: (I)+(O.28.67), B-669: (I)+(O.28.68), B-670: (I)+(O.28.69), B-671: (I)+(O.28.70), B-672: (I)+(O.28.71), B-673: (I)+(O.28.72), B-674: (I)+(O.28.73), B-675: (I)+(O.28.74), B-676: (I)+(O.28.75), B-677: (I)+(O.28.76), B-678: (I)+(O.28.77), B-679: (I)+(O.28.78), B-680: (I)+(O.28.79), B-681: (I)+(O.28.80), B-682: (I)+(O.28.81), B-683: (I)+(O.28.82).

The mixtures of active substances can be prepared as compositions comprising besides the active ingredients at least one inert ingredient (auxiliary) by usual means, e. g. by the means given for the compositions of compounds I.

Concerning usual ingredients of such compositions reference is made to the explanations given for the compositions containing compounds I.

The mixtures of active substances according to the present invention are suitable as fungicides, as are the compounds of formula I. They are distinguished by an outstanding effectiveness against a broad spectrum of phytopathogenic fungi, especially from the classes of the Ascomycetes, Basidiomycetes, Deuteromycetes and Peronosporomycetes (syn. Oomycetes). In addition, it is refered to the explanations regarding the fungicidal activity of the compounds and the compositions containing compounds I, respectively.

### I. SYNTHESIS

### Example 1: This Example Illustrates the Preparation of N-(1-bicyclo[1.1.1]pentanyl)-4-[5-(trifluoromethyl)-1,2,4-oxadiazol-3-yl]benzamide (Ex-1)

I.1) Preparation of 4-[(Z)—N′-hydroxycarbamimidoyl]benzoic acid To a solution of the 4-cyano benzoic acid (500 g, 1.0 eq.) in a mixture of ethanol and water (5 liter/2 liter) was added hydroxylamine hydrochloride (495 g, 2.0 eq.) and potassium carbonate (751 g, 1.5 eq.). To the resulting mixture 8-hydroxyquinoline (6.5 g, 0.1 eq.) was added and it was heated under reflux until HPLC indicated complete conversion of the starting material. After cooling to ambient temperature, water was added and the resulting precipitate was collected and dried to afford the title compound sufficiently pure to be used directly without further purification.

### I.2) Preparation of 4-[5-(trifluoromethyl)-1,2,4-oxadiazol-3-yl]benzoic acid

A solution of 4-[(Z)—N′-hydroxycarbamimidoyl]benzoic acid (200 g, 1.0 eq.) in tetrahydrofurane (2.5 liter) was treated with trifluoroacetic anhydride (350 g, 1.5 eq.). The resulting mixture was stirred overnight at ambient temperature, before it was diluted with methyl tert-butyl ether and washed with a saturated aqueous solution of sodium bicarbonate. The organic layer was removed under reduced pressure to afford a crude product that was recrystallized from di-iso-propyl ether to furnish the title compound as light brown solid (220 g, 76%). 1H NMR (DMSO-d₆, 400 MHz): δ [ppm]=13.40 (br. s, 1H), 8.22-8.10 (m, 4H).

### I.3) Preparation of N-(1-bicyclo[1.1.1]pentanyl)-4-[5-(trifluoromethyl)-1,2,4-oxadiazol-3-yl]benzamide (Ex-1)

To a solution of 4-[5-(trifluoromethyl)-1,2,4-oxadiazol-3-yl]benzoic acid (1.0 g, 4.0 mmol) in tetrahydrofuran (15 mL) Bis(2-oxo-3-oxazolidinyl)phosphinic chloride (1.0 g, 4.0 mmol), triethylamine (0.98 g, 10 mmol) and bicyclo[1.1.1]pentan-1-amine (0.695 g, 6 mmol) was added. After stirring overnight, water was added and the mixture was extracted with tert-butyl methyl ether. The organic layers were separated and washed with brine and dried over sodium sulfate. The solvent was removed under reduced pressure and the crude residue was purified by flash chromatography over silica gel to give the title compound as a colorless solid (0.5 g, 40% yield). ¹H NMR (CDCl₃, 400 MHz): 8.18 (d, 2H), 7.89 (d, 1H), 6.55 (s, 1H), 2.53 (s, 1H), 1.23 (s, 6H).

### Example 2: this Example Illustrates the Preparation of N-[[4-[5-(trifluoromethyl)-1,2,4-oxadiazol-3-yl]phenyl]methyl]norbornane-2-carboxamide (Ex-2)

### I.4) Preparation of N-[(4-cyanophenyl)methyl]-N-methyl-norbornane-2-carboxamide

A round-bottom flask was charged with norbornane-2-carboxylic acid (1.0 g, 1 eq.) and thionyl chloride (O.95 g, 2 eq.) was added dropwise. The mixture was warmed to 70° C. for 1 hour. The mixture was cooled to room temperature and dichloromethane (20 mL) was added. Subsequently 4-(methylaminomethyl)benzonitrile (1.03 g, 1.1 eq) was added portion wise. The mixture was stirred at room temperature over night until HPLC indicated complete conversion. The organic layers were washed with hydrochloric acid (1 M), brine and dried over sodium sulfate. The solvent was removed under reduced pressure and the title compound was used directly without further purification.

### I.5) Preparation of N-[[4-[(Z)—N-hydroxycarbamimidoyl]phenyl]methyl]-N-methyl-norbornane-2-carboxamide

A solution of N-[(4-cyanophenyl)methyl]-N-methyl-norbornane-2-carboxamide obtained from I.4) (0.9 g, 1 eq.) and trimethylamine (0.85 g, 2.5 eq.) in ethanol (10 mL) hydroxylamine hydrochloride (0.47 g, 2 eq.) was added. The mixture was stirred at room temperature. The solvent was removed under reduced pressure. The title compound was used directly without further purification.

### I.6) Preparation of N-[[4-[5-(trifluoromethyl)-1,2,4-oxadiazol-3-yl]phenyl]methyl]norbornane-2-carboxamide (Ex-2)

A solution of N-[[4-[(Z)—N-hydroxycarbamimidoyl]phenyl]methyl]-N-methyl-norbornane-2-carboxamide obtained from I.5) (2.0 g, 1 eq.) in dichloromethane (20 mL) was treated with trifluoroacetic anhydride (7.0 g, 5 eq.). The resulting mixture was stirred overnight at ambient temperature, before it was diluted with water and washed with dichloromethane. The combined organic layer was washed with aqueous solutions of sodium bicarbonate and dried over sodium sulfate. The solvent was removed under reduced pressure and the crude residue was purified by flash chromatography over silica gel to give the title compound (0.6 g, 25% yield over 3 steps). LC/MS (*HPLC method described below): Retention time 1.40 min (m/z 379.9).

The compounds listed in Table I were prepared in an analogous manner.

TABLE I
Compounds Ex-1 to Ex-5 of the formula I.Ex, wherein the meaning of the variables
are as defined in each line, whereas radical R¹ is further unsubstituted; and wherein #1 in
group W denotes the position, which is attached to the group —CR³R⁴— or, if m is 0, to the
phenyl group; and wherein “#-” in R¹ indicates the ring carbon atom, which is attached to the
group W.
I.Ex

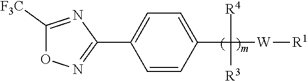

| | | | | | | Melting |
|---|---|---|---|---|---|---|
| Ex. | | | | | HPLC Rₜ | point |
| no | m | R³, R⁴ | W | R¹ | (min)* | (° C.) |
| Ex-1 | 0 | — | #1-(C═O)NH— | R¹.33 | 1.19 | 185-193 |
| Ex-2 | 1 | H, H | #1-NCH₃(C═O)— | 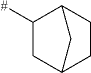 | 1.40 | — |
| Ex-3 | 1 | H, H | #1-NCH₃(C═O)— | 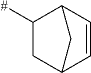 | 1.36 | — |
| Ex-4 | 1 | H, H | #1-NCH₃(C═O)— | 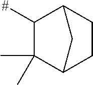 | 1.52 | — |
| Ex-5 | 1 | H, H | #1-NCH₃(C═O)— | R¹.42 | 1.30 | — |

*HPLC: High Performance Liquid Chromatography; HPLC-column Kinetex XB C18 1.7μ (50 × 2.1 mm); eluent: acetonitrile/water + 0.1% trifluoroacetic acid (gradient from 5:95 to 100:0 in 1.5 min at 60° C., flow gradient from 0.8 to 1.0 ml/min in 1.5 min).
MS: Quadrupol Electrospray Ionisation, 80 V (positive mode).
Rₜ: retention time in minutes.

### II. BIOLOGICAL EXAMPLES FOR FUNGICIDAL ACTIVITY

The fungicidal action of the compounds of formula I was demonstrated by the following experiments: The spray solutions were prepared in several steps: The stock solution were prepared by mixting acetone and/or dimethylsulfoxide and the wetting agent/emulsifier Wettol, which is based on ethoxylated alkylphenoles, in a relation (volume) solvent-emulsifier of 99 to 1. This mixture was added to 25 mg of the compound to give a total of 5 mL. Water was then added to total volume of 100 mL. This stock solution was diluted with the described solvent-emulsifier-water mixture to the given concentration.

### II.1) Curative Control of Soy Bean Rust on Soy Beans Caused by Phakopsora pachyrhizi

Leaves of pot-grown soy bean seedlings were inoculated with spores of Phakopsora pachyrhizi. To ensure the success of the artificial inoculation, the plants were transferred to a humid chamber with a relative humidity of about 95% and 20 to 24° C. for 24 hours. The next day the plants were cultivated for 3 days in a greenhouse chamber at 23 to 27° C. and a relative humidity between 60 and 80%. Then the plants were sprayed to run-off with an aqueous suspension, containing the concentration of active ingredient or their mixture as described below. The plants were allowed to air-dry. Then the trial plants were cultivated for 14 days in a greenhouse chamber at 23 to 27° C. and a relative humidity between 60 and 80%. The extent of fungal attack on the leaves was visually assessed as % diseased leaf area.

In this test, the plants which had been treated with 63 ppm of the active compounds Ex-3 and Ex-5 showed a diseased leaf area of at most 1%, whereas the untreated plants showed 100% diseased leaf area.

In this test, the plants which had been treated with 32 ppm of the active compound Ex-1 showed a diseased leaf area of 0%, whereas the untreated plants showed 100% diseased leaf area.

### II.2) Protective Control of Soy Bean Rust on Soy Beans Caused by Phakopsora pachyrhizi

Leaves of pot-grown soy bean seedlings were sprayed to run-off with an aqueous suspension, containing the concentration of active ingredient or their mixture as described below. The plants were allowed to air-dry. The trial plants were cultivated for 2 day in a greenhouse chamber at 23 to 27° C. and a relative humidity between 60 and 80%.Then the plants were inoculated with spores of Phakopsora pachyrhizi. To ensure the success the artificial inoculation, the plants were transferred to a humid chamber with a relative humidity of about 95% and 20 to 24° C. for 24 hours. The trial plants were cultivated for fourteen days in a greenhouse chamber at 23 to 27° C. and a relative humidity between 60 and 80%. The extent of fungal attack on the leaves was visually assessed as % diseased leaf area.

In this test, the plants which had been treated with 63 ppm of the active compounds Ex-2, Ex-3, Ex-4 and Ex-5 showed a diseased leaf area of at most 6%, whereas the untreated plants showed 90% diseased leaf area.

In this test, the plants which had been treated with 32 ppm of the active compound Ex-1 showed a diseased leaf area of 0%, whereas the untreated plants showed 100% diseased leaf area.

### II.3) Curative Control of Brown Rust on Wheat Caused by Puccinia Recondite

The first two developed leaves of pot-grown wheat seedling were dusted with spores of Puccinia recondite. To ensure the success the artificial inoculation, the plants were transferred to a humid chamber without light and a relative humidity of 95 to 99% and 20 to 24° C. for 24 hours. The next day the plants were cultivated for 3 days in a greenhouse chamber at 20 to 24° C. and a relative humidity between 65 and 70%. Then the plants were sprayed to run-off with an aqueous suspension, containing the concentration of active ingredient or their mixture as described below. The plants were allowed to air-dry. Then the trial plants were cultivated for 8 days in a greenhouse chamber at 20 to 24° C. and a relative humidity between 65 and 70%. The extent of fungal attack on the leaves was visually assessed as % diseased leaf area.

In this test, the plants which had been treated with 63 ppm of the active compound Ex-5 showed a diseased leaf area of 0%, whereas the untreated plants showed 90% diseased leaf area.

In this test, the plants which had been treated with 32 ppm of the active compound Ex-1 showed a diseased leaf area of 2%, whereas the untreated plants showed 90% diseased leaf area.

1-14. (canceled)

15. A compound of formula I, or an N-oxide, or an agriculturally acceptable salt thereof, 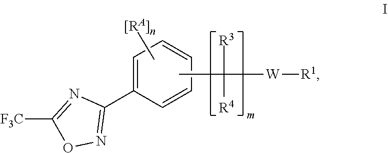
wherein:
R^{A} is independently selected from the group consisting of halogen, cyano, C₁-C₆-alkyl, C₁-C₆-haloalkyl, C₁-C₆-alkoxy and C₁-C₆-haloalkoxy;
n is 0, 1 or 2;
R¹ is a bicyclic carbocycle of the formula R^{a} 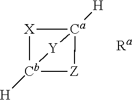
wherein
C^{a} and C^{b} are bridgehead carbon atoms;
X is a direct single bond or a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—;
Y and Z independently of each other are a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—;
or R¹ is a tricyclic carbocycle of the formula R^{b} 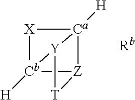
wherein
C^{a} and C^{b} are bridgehead carbon atoms;
X is a direct single bond or a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—;
Y and Z independently of each other are a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—; and wherein groups Y and Z are attached to the bridgehead carbon atoms C^{a} and C^{b};
T is a divalent group selected from the group consisting of —CH₂—, —CH₂—CH₂—, —(CH₂)₃—, —(CH₂)₄—, —CH═CH—, —CH₂—CH═CH—, —CH═CH—CH₂— and —CH═CH—CH═CH—; and wherein the group T is attached to one carbon atom in each of the groups Y and Z;
and with the proviso that, if R¹ is a tricyclic carbocycle of the formula R^{b},
wherein X is a direct single bond or a divalent group —CH₂—, the groups T and Z independently of each other are a divalent group selected from the group consisting of —CH₂—CH₂—, —(CH₂)₃—, —(CH₂)₄—, —CH═CH—, —CH₂—CH═CH—, —CH═CH—CH₂— and —CH═CH—CH═CH—;
and wherein the groups R^{a} or R^{b} are connected to the group W through one of the ring carbon atoms;
and wherein R¹ is unsubstituted or substituted with 1, 2, 3, 4 or up to the maximum possible number of radicals selected from the group consisting of oxo, hydroxy, halogen, C₁-C₃-alkyl, C₁-C₃-haloalkyl, C₃-C₆-cycloalkyl, vinylidene and dichlorovinylidene;
W is #1-(C═O)—NR²-#2, #1-NR²—(C═O)-#2, #1-(C═S)—NR²-#2, #1-NR²—(C═S)-#2, #1-S(═O)ₚ—NR²-#2 or #1-NR²—S(═O)ₚ-#2; wherein #1 denotes the position, which is attached to the group —CR³R⁴— or, if m is 0, to the phenyl group, and #2 denotes the position, which is attached to R¹;
p is 0, 1 or 2;
R² is hydrogen, C₁-C₆-alkyl, C₂-C₆-alkenyl, C₂-C₆-alkynyl, C₁-C₆-alkoxy, C₃-C₈-cycloalkyl, C₃-C₈-cycloalkenyl, C₃-C₈-cycloalkyl-C₁-C₄-alkyl, phenyl-C₁-C₄-alkyl, phenyl, C(═O)—(C₁-C₆-alkyl) or C(═O)—(C₁-C₆-alkoxy); and wherein any of the aliphatic or cyclic groups are unsubstituted or substituted with 1, 2, 3 or up to the maximum possible number of identical or different radicals selected from the group consisting of halogen, hydroxy, cyano, C₁-C₆-alkyl, C₁-C₆-alkoxy and C₃-C₈-cycloalkyl;
m is 0 or 1;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy;
or R³ and R⁴ together with the carbon atom to which they are bound form a monocyclic 3- to 5-membered saturated heterocycle or saturated carbocycle; and wherein the saturated heterocycle includes beside one or more carbon atoms no heteroatoms or 1 or 2 heteroatoms independently selected from the group consisting of N, O and S as ring member atoms; and wherein the heterocycle or the carbocycle is unsubstituted or substituted 1, 2, 3, 4 or up to the maximum possible number of identical or different radicals selected from the group consisting of halogen, cyano and C₁-C₂-alkyl;
with the exception of compounds of the formula I, wherein
m is 0;
W is #1-NR²—(C═O)-#2, #1-NR²—(C═S)-#2 or #1-NR²—S(═O)ₚ-#2, wherein the variables p and R² are defined as above for compounds of the formula I; and
R¹ is a bicyclic carbocycle of the formula R^{a}.

16. The compound of claim 15 having formula 1.3, 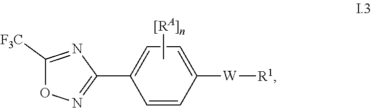
wherein n is 0 or 1, and wherein the meaning of the variables R^{A}, R¹, W and R² are as defined for compounds of the formula I in claim 15.

17. The compound of claim 15, wherein n is 0.

18. The compound of claim 15, wherein W is #1-(C═O)—NR²-#2, #1-(C═S)—NR²-#2 or #1-S(═O)ₚ—NR²-#2.

19. The compound of claim 15, wherein R³ and R⁴ independently of each other are selected from the group consisting of hydrogen, halogen, C₁-C₆-alkyl and C₁-C₆-haloalkyl; or R³ and R⁴ together with the carbon atom to which they are bound form a cyclopropyl ring.

20. The compound of claim 15, wherein R¹ is a bicyclic carbocycle selected from the group consisting of radicals R¹.10, R¹.15, R¹.22, R¹.23, R¹.24, R¹.25, R¹.26, R¹.27 and R¹.31 as defined herein; and wherein each radical may be connected to the group W through one of the ring carbon atoms by substitution of one hydrogen atom; and wherein R¹ is unsubstituted or substituted with 1, 2, 3, 4 or up to the maximum possible number of radicals selected from the group consisting of oxo, hydroxy, halogen and C₁-C₃-alkyl.

21. The compound of claim 15, wherein R¹ is a bicyclic carbocycle selected from the group consisting of radicals R¹.32, R¹.33, R¹.34 and R¹.35 as defined herein; and wherein R¹ is unsubstituted or substituted with 1, 2, 3, 4 or up to the maximum possible number of radicals selected from the group consisting of oxo, hydroxy, halogen and C₁-C₃-alkyl.

22. The compound of claim 15, wherein R¹ is unsubstituted or substituted with 1, 2, 3, 4 or up to the maximum possible number of radicals selected from the group consisting of hydrogen, fluorine, chlorine, methyl and ethyl.

23. The compound of claim 15, wherein R² is hydrogen, C₁-C₆-alkyl, C₁-C₆-alkenyl, ethynyl, propargyl, C₃-C₈-cycloalkyl, C₃-C₈-cycloalkyl-C₁-C₄-alkyl or phenyl; and wherein any of the aliphatic or cyclic groups are unsubstituted or substituted with 1, 2, 3, 4 or up to the maximum possible number of identical or different radicals selected from the group consisting of halogen and C₁-C₆-alkyl.

24. An agrochemical composition which comprises an auxiliary and at least one compound of the formula I, or an N-oxide, or an agriculturally acceptable salt thereof, as defined in claim 15.

25. The agrochemical composition of claim 24, wherein the auxiliary is selected from the group consisting of ionic and non-ionic surfactants.

26. The agrochemical composition claim 24, further comprising seed, wherein the amount of the compound of the formula I, or an N-oxide, or an agriculturally acceptable salt thereof, is from 0.1 g to 10 kg per 100 kg of seed.

27. A method for combating phytopathogenic harmful fungi, which process comprises treating the fungi, the plants, the soil or seeds to be protected against fungal attack, with an effective amount of at least one compound of formula I, or an N-oxide, or an agriculturally acceptable salt thereof, as defined in claim 15.

28. The method of claim 27, wherein the compound of formula I is compound of formula 1.3, 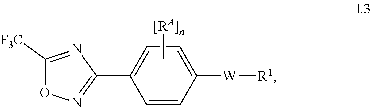
wherein n is 0 or 1, and wherein the meaning of the variables R^{A}, R¹, W and R² are as defined for compounds of the formula I in claim 15.

29. The method of claim 27, wherein n is 0.

30. The method of claim 27, wherein W is #1-(C═O)—NR²-#2, #1-(C═S)—NR²-#2 or #1-S(═O)ₚ—NR²-#2.

31. The method of claim 27, wherein R³ and R⁴ independently of each other are selected from the group consisting of hydrogen, halogen, C₁-C₆-alkyl and C₁-C₆-haloalkyl; or R³ and R⁴ together with the carbon atom to which they are bound form a cyclopropyl ring.

32. The method of claim 27, wherein R¹ is a bicyclic carbocycle selected from the group consisting of radicals R¹.10, R¹.15, R¹.22, R¹.23, R¹.24, R¹.25, R¹.26, R¹.27 and R¹.31 as defined herein; and wherein each radical may be connected to the group W through one of the ring carbon atoms by substitution of one hydrogen atom; and wherein R¹ is unsubstituted or substituted with 1, 2, 3, 4 or up to the maximum possible number of radicals selected from the group consisting of oxo, hydroxy, halogen and C₁-C₃-alkyl.

33. The method of claim 27, wherein R¹ is a bicyclic carbocycle selected from the group consisting of radicals R¹.32, R¹.33, R¹.34 and R¹.35 as defined herein; and wherein R¹ is unsubstituted or substituted with 1, 2, 3, 4 or up to the maximum possible number of radicals selected from the group consisting of oxo, hydroxy, halogen and C₁-C₃-alkyl.

34. The method of claim 27, wherein R¹ is unsubstituted or substituted with 1, 2, 3, 4 or up to the maximum possible number of radicals selected from the group consisting of hydrogen, fluorine, chlorine, methyl and ethyl.

35. The method of claim 27, wherein R² is hydrogen, C₁-C₆-alkyl, C₁-C₆-alkenyl, ethynyl, propargyl, C₃-C₈-cycloalkyl, C₃-C₈-cycloalkyl-C₁-C₄-alkyl or phenyl; and wherein any of the aliphatic or cyclic groups are unsubstituted or substituted with 1, 2, 3, 4 or up to the maximum possible number of identical or different radicals selected from the group consisting of halogen and C₁-C₆-alkyl.
